(19) United States
(12) Patent Application Publication
(10) Pub. No.: US 2014/0243217 A1
(43) Pub. Date: Aug. 28, 2014

(54) METHODS AND COMPOSITIONS FOR DETECTING PLANT EXPOSURE TO PLANT PATHOGENS

(71) Applicant: Illimar Altosaar, Ottawa (CA)

(72) Inventor: Illimar Altosaar, Ottawa (CA)

(73) Assignee: University of Ottawa

(21) Appl. No.: 13/817,598

(22) PCT Filed: Nov. 4, 2011
(86) PCT No.: PCT/CA2011/050688

§ 371 Date: Feb. 19, 2013

Related U.S. Application Data
(60) Provisional application No. 61/552,258, filed on Oct. 27, 2011.

(51) Int. Cl. C12Q 1/37

(52) U.S. Cl. CPC C12Q 1/37
USPC 506/6; 436/89; 435/23; 435/34

(57) ABSTRACT

The present disclosure provides methods an compositions for the detection of plant exposure to plant pathogens. The methods relate to determining the identity of peptides isolated from the surface of plant starch granules.

### SEQUENCE LISTING

The present application contains a Sequence Listing which has been submitted in ASCII format via EFS-Web and is hereby incorporated by reference in its entirety. The ASCII copy, created on Sep. 27, 2011, is named 09161906.txt and is 28,187 bytes in size.

### FIELD OF TECHNOLOGY

The present technology relates to methods for detecting the exposure of plants to plant pathogens. In particular, the methods relate to identifying peptides isolated from the surface of plant starch granules which are indicative of exposure to plant pathogens.

### BACKGROUND

Plants capture energy from sunlight and convert it into vitreous amyloplast-derived starch granule deposits composed of amylose and amylopectin. These granules can be transitory, as found in green tissues, or stored long-term, such as in reserve tissues of stems, tubers, fruits, nuts and grain endosperm. Fungal and bacterial plant pathogens have adapted to attack starch granules and draw vital nutrition from their energy stores. Host resistance involves organelle-specific innate defense mechanisms and antimicrobial small molecules. Proteins and peptides at the surface of the starch granule can be considered the final line of defense against the invading organisms. Therefore, the starch granule surface represents a subcellular compartment where one may observe not only the direct interactions of host molecular defense mechanisms, but also the pathogen molecular attack mechanisms.

### SUMMARY

In one aspect, the present disclosure relates generally to methods for detecting the exposure of a non-grain plant or plant part to at least one plant pathogen. In illustrative embodiments, the methods comprise isolating at least one starch granule from the non-grain, contacting the starch granule with at least one solvent, wherein the contacting releases at least one peptide from the surface of the starch granule, and determining the amino acid sequence of the peptide. In illustrative embodiments, the presence of at least one peptide from a plant pathogen indicates that the non-grain has been exposed to at least one plant pathogen.

In illustrative embodiments, the non-grain comprises fruits, nuts, vegetables, legumes, tubers, leaves, trees, flowers, herbs, bushes, grasses, vines, ferns, mosses, fungi, petals, flour, anthers, pollen, pollen grains, glumes, sugarcane stems, awns, straw, grain dust, palea, pedicels, rachilla, lodicules, stolons, rhizomes, sheaths, collars, blades, auricles, ligules, culms, or green algae. In illustrative embodiments, the solvent comprises at least one protease. In illustrative embodiments, the protease comprises trypsin or chymotrypsin.

In illustrative embodiments, the at least one plant pathogen comprises a viral pathogen, a bacterial pathogen, or a fungal pathogen. In illustrative embodiments, the at least one plant pathogen comprises Agrobacterium, Pectobacterium, Fusarium, Magnaporthe, or Xanthomonas. In illustrative embodiments, the at least one plant pathogen comprises Agrobacterium tumefaciens, Pectobacterium carotovorum, Fusarium graminearum, Magnaporthe grisea, Xanthomonas axonopodis, or Xanthomonas oryzae. In illustrative embodiments, the determining step comprises chromatography, mass spectrometry, or both.

In illustrative embodiments, the amino acid sequences of the peptide comprise at least one amino acid sequence selected from the group consisting of amino acid sequences listed in Tables 1-5.

In another aspect, the present disclosure provides a method for detecting a response to at least one plant pathogen in a non-grain plant or plant part, the method comprising isolating at least one starch granule from the non-grain, contacting the starch granule with at least one solvent, wherein the contacting releases at least one peptide from the surface of the starch granule, and determining the amino acid sequence of the peptide. In illustrative embodiments, the presence of at least one peptide from non-grain anti-microbial proteins indicates a non-grain response to at least one plant pathogen.

In illustrative embodiments, the non-grain comprises fruits, nuts, vegetables, legumes, tubers, leaves, trees, flowers, herbs, bushes, grasses, vines, ferns, mosses, fungi, or green algae. In illustrative embodiments, the solvent comprises at least one protease. In illustrative embodiments, the protease comprises trypsin or chymotrypsin.

In illustrative embodiments, the at least one plant pathogen comprises a viral pathogen, a bacterial pathogen, or a fungal pathogen. In illustrative embodiments, the at least one plant pathogen comprises Agrobacterium, Pectobacterium, Fusarium, Magnaporthe, or Xanthomonas. In illustrative embodiments, the at least one plant pathogen comprises Agrobacterium tumefaciens, Pectobacterium carotovorum, Fusarium graminearum, Magnaporthe grisea, Xanthomonas axonopodis, or Xanthomonas oryzae. In illustrative embodiments, the determining step comprises chromatography, mass spectrometry, or both.

In illustrative embodiments, the amino acid sequences of the peptide comprise at least one amino acid sequence selected from the group consisting of amino acid sequences listed in Tables 1-5.

In another aspect, the present disclosure provides a kit for detecting the exposure of a non-grain plant or plant part to at least one plant pathogen, the kit comprising at least one solvent, wherein the at least one solvent extracts peptides present on the surface of at least one starch granule isolated from the non-grain, at least one negative control sample, comprising at least one starch granule from a pest-free, pathogen-free non-grain, and at least one positive control, comprising at least one starch granule isolated from a plant exposed to a plant pathogen.

In illustrative embodiments, the negative control sample comprises starch from a plant resistant to Fusarium. In illustrative embodiments, the positive control sample comprises starch from a plant contacted with Fusarium. In illustrative embodiments, the positive control sample comprises at least one peptide having an amino acid sequence selected from the group consisting of amino acid sequences listed in Tables 1-5. In illustrative embodiments, the solvent comprises trypsin or chymotrypsin.

In another aspect, the present disclosure provides a processing circuit for determining the presence of a plant pathogen in a non-grain plant or plant part. The processing circuit includes one or more processors and one or more memory devices. The one or more memory devices store amino acid data indicative of plant pathogens or non-grain anti-microbial plant proteins. The one or more memory devices also store instructions that, when executed by the one or more processors, cause the one or more processors to execute operations. The operations include receiving data indicative of a peptide sequence of a plant pathogen or anti-microbial plant protein. The operations also include determining the presence of a pathogen by comparing the received data to the stored amino acid data. The operations further include providing an indication of the determination to an electronic device. The amino acid data may include data indicative of one or more amino acid sequences selected from the group consisting of amino acid sequences listed in Tables 1-5.

In a further aspect, the present disclosure provides a computerized method for detecting the exposure of a non-grain plant or plant part to at least one plant pathogen. The method includes storing, in one or more memory devices, data indicative of one or more amino acid sequences. In illustrative embodiments, the amino acid sequences may be selected from the group consisting of amino acid sequences listed in Tables 1-5. The method also includes receiving data indicative of a peptide sequence of a plant pathogen or anti-microbial plant protein. The method further includes determining, by a processing circuit, the presence of a pathogen by comparing the received data to the stored amino acid data. The method also includes providing an indication of the determination to an electronic device.

### BRIEF DESCRIPTION OF THE DRAWINGS

FIG. 1. SDS-PAGE separation of peptides isolated from Fusarium-infected Site 431 and Site 388 corn kernels. 4% stacking and 15% resolving gel run at 15 mA. Outside lanes (MM): protein molecular weight markers, 14.4-116 kDa (Fermentas, Burlington, Ontario, CA; cat #SM0431; 5 μL loaded). Lane 1: Site 431, control; Lane 2: Site 431, F. verticillium-infected; Lane 3: Site 431, F. graminearum-infected; Lane 4: empty; Lane 5: Site 388, control; Lane 6: Site 388, F. verticillium-infected; Lane 7: Site 388, F. graminearum-infected. The arrows (<) indicate fungal protein bands present in the F. verticillium and F. graminearum kernel extracts but not in the control sample.

FIG. 2. SDS-PAGE separation of peptides isolated from Fusarium-infected Site 359 and Site C130 corn kernels. 4% stacking and 15% resolving SDS-PAGE gel run at 15 mA. Outside lanes (MM): protein molecular weight markers, 6.5 to 66 kDa. (Sigma-Aldrich; St. Louis, Mo., USA; cat #M3913; 5 μL loaded). Lane 1: Site 359, control; Lane 2: Site 359, F. verticillium-infected; Lane 3: Site 359, F. graminearum-infected; Lane 4: empty; 5: Site C130, control; Lane 6: Site C130, F. verticillium-infected; Lane 7: Site C130, F. graminearum-infected. The arrows (<) indicate fungal protein bands present in the F. verticillium and F. graminearum kernel extracts but not in the control sample.

### DETAILED DESCRIPTION

In the following detailed description, reference may be made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present technology relates to methods for detecting the exposure of a plant or plant product to one or more plant pathogens, comprising isolating peptides from the surface of at least one starch granule, and determining the identity of the peptides. The present technology further relates to methods for detecting the response of a plant to one or more plant pathogens, comprising isolating peptides from the surface of at least one plant starch granule, and determining the identity of the peptides. In illustrative embodiments, the plant comprises a photosynthetic plant or a non-photosynthetic plant. In illustrative embodiments, the plant comprises a monocot or a dicot. In illustrative embodiments, the plant comprises a grain. In illustrative embodiments, the grain comprises maize, rice, wheat, barley, sorghum, millet, oats, rye, soybeans, canola, triticale, fonio, buckwheat, quinoa, teff, rice, wild rice, amaranth, kaniwa, spelt, einkorn, emmer, and durum. In illustrative embodiments, the grain comprises the genus Triticum. In illustrative embodiments, the starch granule comprises the endosperm of a grain seed.

In illustrative embodiments, the plant comprises a non-grain. In illustrative embodiments, the non-grain comprises fruits, nuts, vegetables, legumes, tubers, leaves, trees, flowers, herbs, bushes, grasses, vines, ferns, mosses, fungi, or green algae.

In illustrative embodiments, the starch granule comprises transient storage starch. In illustrative embodiments, the starch granule comprises long-term storage starch. In illustrative embodiments, the starch comprises starch stored within inclusion bodies. In illustrative embodiments, the starch comprises starch stored within amyloplasts and chloroplasts. In illustrative embodiments, the starch comprises starch stored amorphously outside of amyloplasts and chloroplasts. In illustrative embodiments, the starch comprises amylose and amylopectin.

The starch granule proteome recovered from plants at various stages of the agri-industrial food chain, including grain elevators, train cars and cargo holds of ships, can serve as an indisputable ‘molecular’ passport or report card of the plant's phytosanitary conditions, quality and legal classification.

The present technology further relates to kits for detecting the exposure of a plant to one or more plant pathogens and/or detecting the response of a plant to one or more plant pathogens. In some embodiments, the kit comprises at least one solvent, at least one negative control sample, and at least one positive control sample. The present technology further relates to a database comprising amino acid sequences indicative of plant pathogens, such as amino acids from plant pathogens, and from plant pathogen-response proteins. Such a database may be used, for example, as part of a processing circuit configured to determine the presence of a plant pathogen.

In illustrative embodiments, the presence of one or more peptides described herein on the surface of a plant starch granule indicates that the plant has been exposed to a plant pathogen. In illustrative embodiments, the presence of such peptides indicates an active infection by a plant pathogen. In illustrative embodiments, the presence of such peptides indicates a past infection by a plant pathogen. In illustrative embodiments, the presence of such peptides indicates exposure to a plant pathogen that did not result in infection of the plant. In illustrative embodiments, the peptides comprise one or more of the amino acid sequences listed in Tables 1-5.

In practicing the present methods, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Plant Infectious Agents: Viruses, Viroids, Virusoids, and Satellites, Robertson, Ed. (Cold Spring Harbor Laboratory Press, Cold Spring Harbor, N.Y., 1983); Methods in Plant Molecular Biology: A Laboratory Course Manual, Maliga, Ed. (Cold Spring Harbor Laboratory Press, Cold Spring Harbor, N.Y., 1995), and Plant Biology (Garland Science, 2010). Methods to detect and measure levels of peptides in a sample are known in the art, such as high performance liquid chromatography and mass spectrometry.

The definition of certain terms used herein are provided below. Unless defined otherwise, all technical and scientific terms used herein have the same meaning as commonly understood by one of ordinary skill in the art to which the present disclosure pertains.

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry, biochemistry, and nucleic acid chemistry and hybridization described below are those well known and commonly employed in the art. All references cited herein are incorporated by reference in their entirety for all purposes to the same extent as if each individual publication, patent, or patent application were specifically and individually incorporated by reference in its entirety for all purposes.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the term “grain” refers to the seed portion of cereal and grass monocot plant family Poaceae cultivated for the edible components of their “grain” (caryopsis) comprising endosperm, germ, and bran. By way of example, but not by way of limitation, grains include maize, rice, wheat, barley, sorghum, millet, oats, rye, triticale, fonio, buckwheat, quinoa, teff, wild rice, amaranth, kaniwa, spelt, einkorn, emmer, and durum. In illustrative embodiments, the grain comprises the genus Triticum. In illustrative embodiments, grain comprises Triticum turgidum durum or Triticum aestivum. As used herein, the term “grain” does not encompass portions of monocot plants other than the caryopsis, such as leaves stems and roots.

The term “non-grain” refers to plants and plant parts not encompassed by the definition of “grain” set forth above. As used herein the term includes but is not limited to fruits, nuts, vegetables, legumes, tubers, leaves, trees, flowers, herbs, bushes, grasses, vines, ferns, mosses, fungi, and green algae. As used herein, the term encompasses the non-caryopsis portion of plants traditionally cultivated as cereal grains, such as those described above, including but not limited to leaves, stems, and roots. As used herein, non-grains includes plants cultivated for consumption, landscaping, interior design, construction, and fuel. The term encompasses non-grains cultivated, for example, for the production of food, food additives, pharmaceuticals, dietary supplements, cosmetics, herbicides, insecticides, fertilizer, pigments, paints, inks, dyes, varnishes, waxes, oils, adhesives, wood products, textiles, paper, etc.

As used herein, the term “hypothetical peptide” or “hypothetical protein” refers to a peptide or protein whose existence has been predicted, but for which there is yet no empirical evidence of expression in vivo. In illustrative embodiments, peptides extracted from the surface of starch granules are hypothetical peptides.

As used herein, the term “starch” refers to a carbohydrate comprising glucose units joined together by glycosidic bonds produced and stored by plants as an energy reserve. As used herein, the term encompasses linear and helical amylose and branched amylopectin, and encompasses starch stored in plastids such as chloroplasts and amyloplasts. In illustrative embodiments, starch comprises α-1, 4, or α-1,6-glucan polymers. In illustrative embodiments, starch comprises non-aggregated and non-coalesced starch, and starch that accumulates amorphously outside of organelles. In illustrative embodiments, the starch comprises transitory storage starch and long-term storage starch. As used herein, the term encompasses starch stored in fruit, seeds, kernels, cobs, husks, rhizomes, tubers, fronds, leaves, stems, barks, roots, petals, flour, anthers, pollen, pollen grains, glumes, sugar cane stems, awns, straw, palea, pedicels, rachilla, lodicules, stolons, rhizomes, sheaths, collars, blades, auricles, ligules, culms, and twigs of a plant. In illustrative embodiments, starch comprises the endosperm starch of a seed. In illustrative embodiments, the starch comprises the endosperm starch of a grain.

As used herein, “plant disease” refers to any condition that alters the appearance, viability, regulatory classification (e.g., grade), commercial value, or productivity of a plant, including but not limited to alterations in rate of growth, photosynthesis, flowering, or seed production. The term also encompasses conditions that cause aberrations in the appearance of a plant such as aberrations in color, size, or shape, irrespective of the effect on plant viability. Disease conditions may effect plant root systems, foliage, or both. By way of example, but not by way of limitation, plant diseases include crown rust, stem rust, blast, blight, potato late blight, Phytophthora, smut, Claviceps purpurea, Ustilago, Fusarium wilt disease, canker rot, black root rot, soft rot, Thielaviopsis root rot, gray leaf spot in turfgrasses, sudden oak death, apple scab, and banana blemishes.

As used herein, the term “plant pathogen” refers to organisms that cause disease in plants. The term encompasses mycoplasma, fungi, oomycetes, bacteria, viruses, viroids, virus-like organisms, phytoplasmas, protozoa, nematodes, parasitic plants. Phytopathogenic fungi comprise organisms of the taxa Ascomycetes, Basidiomycetes, and Oomycetes. Organisms of the taxa Ascomycetes, include but are not limited to Fusarium spp., Thielaviopsis spp., and Verticillium spp. Organisms of the taxa Basidiomycetes, include but are not limited to Rhizoctonia spp. (e.g., Phakospora pachyrhizi), and Puccinia spp. Organisms of the taxa Oomycetes, include but are not limited to Pythium spp., and Phytophthora spp. Pathogenic bacteria include but are not limited to Erwinia, Agrobacterium, Burkholderia, Proteobacteria, Xanthomonas spp., Pseudomonas spp., Phytoplasma, mycoplasma-like organisms, Spiroplasma, and mollicutes. Fusarium, Thielaviopsis, Verticillium, Rhizoctonia, Phakospora, Puccinia, Pythium, Phytophthora, Erwinia, Agrobacterium, Burkholderia, Proteobacteria, Xanthomonas, Pseudomonas, Phytoplasma, Spiroplasma, Pectobacterium, or Magnaporthe. In illustrative embodiments, plant pathogens comprise, Agrobacterium tumefaciens, Pectobacterium carotovorum, Fusarium graminearum, Magnaporthe grisea, Xanthomonas axonopodis, or Xanthomonas oryzae.

As used herein, the term “plant pathogen-response (PR) protein” refers to proteins produced by a plant in response to exposure to a plant pathogen. In illustrative embodiments, PR proteins comprise anti-microbial proteins. Illustrative anti-microbial PR proteins include, but are not limited to an amylase inhibitor, a glycosylase inhibitor, a xylanase inhibitor, a peroxidase, and a chitinase. In illustrative embodiments, PR proteins comprise the peptides listed in Tables 2 and 3.

As used herein, the term “solvent” comprises any liquid, gel, solid or gas that dissolves another solid, liquid or gaseous solute. In some embodiments a solvent is present on a substrate such as a paper strip, dip stick, or lateral-flow clinical testing strip that comprises a liquid-phase in its interstitial spaces so that a sample, such as a starch sample, may interact with a solvent phase. As used herein, the term encompasses aqueous and organic solvents. In illustrative embodiments, the solvent comprises an aqueous solution comprising a protease. In illustrative embodiments, the protease comprises trypsin, chymotrypsin, elastase, pepsin, or subtilisin. In illustrative embodiments, the protease comprises a cysteine protease. In illustrative embodiments, the solvent comprises isopropanol. In illustrative embodiments, the solvent comprises an alcohol. In illustrative embodiments, the solvent comprises and alkane. In illustrative embodiments, the solvent comprises ammonium bicarbonate buffer (AmBic), acetic acid or other such volatile buffer salts, dioxane, DMSO, benzene, or pyrimidine.

As used herein, the terms “identical” or percent “identity”, when used in the context of two or more nucleic acids or polypeptide sequences, refers to two or more sequences or subsequences that are the same or have a specified percentage of amino acid residues or nucleotides that are the same (i.e., about 60%, 65%, 70%, 75%, 80%, 85%, 90%, 91%, 92%, 93%, 94%, 95%, 96%, 97%, 98%, 99%, or higher identity over a specified region (e.g., nucleotide sequence encoding a peptide described herein or amino acid sequence of a peptide described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the complement of a test sequence. The term also includes sequences that have deletions and/or additions, as well as those that have substitutions. As described below, the algorithms can account for gaps and the like. In some embodiments, identity exists over a region that is at least about 25 amino acids or nucleotides in length, or 50-100 amino acids or nucleotides in length.

An “isolated” or “purified” polypeptide or biologically-active portion thereof is substantially free of cellular material or other contaminating polypeptides from the cell or tissue source from which the polypeptide is derived, or substantially free from chemical precursors or other chemicals when chemically synthesized. For example, an isolated peptide would be substantially free of materials that would interfere with diagnostic methods used to further purify the peptide or to determine the amino acid sequence of the peptide. Such interfering materials may include enzymes, hormones and other proteinaceous and nonproteinaceous solutes.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, e.g., recombinant cells express genes that are not found within the native (non-recombinant) form of the cell or express native genes that are otherwise abnormally expressed, under expressed or not expressed at all.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature. In illustrative embodiments, peptides of the present technology comprise microbial peptides. In illustrative embodiments, the peptides comprise peptides from plan pathogens. In illustrative embodiments, the peptides comprise plant pathogen-response peptides.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, e.g., recombinant cells express genes that are not found within the native (non-recombinant) form of the cell or express native genes that are otherwise abnormally expressed, under expressed or not expressed at all.

As used herein, the term “amino acid substitution” refers to the replacement of a naturally occurring or wild type amino acid at a given position within a peptide or polypeptide with another residue. Conservative substitutions are those in which substitute amino acids share the same or similar chemical properties as the naturally occurring amino acid, and/or do not alter the biological properties of the peptide or polypeptide as a whole. Non-conservative substitutions are those in which the substitute amino acid does not share the same or similar chemical properties as the naturally occurring amino acid, and/or those which alter the biological properties of the peptide or polypeptide as a whole. For example, one or more amino acid residues within a peptide sequence can be substituted for another amino acid of a similar polarity which acts as a functional equivalent, resulting in a silent alteration (a conservative amino acid substitution). Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. One of skill in the art will understand that methods for selecting alternative amino acids are well known in the art, as are methods for the introduction of amino acid substitutions into a protein or peptide.

As used herein, the term “database” refers to any form of electronic data storage. For example, a database may be one or more data structures, files, memory locations, tables, or any other form of electronic data storage configured to store and retrieve data.

### I. Compositions of the Present Technology

In one aspect, the present technology comprises novel peptides isolated from the surface of a starch granule of a plant. In some embodiments, the peptides are isolated from the surface of a starch granule of a grain. In some embodiments, the peptides are isolated from the surface of a starch granule of a non-grain In illustrative embodiments, the peptides comprise amino acid sequences from plant pathogens. In illustrative embodiments, the peptides comprise amino acid sequences listed in Table 1. In illustrative embodiments, the peptides comprise amino acid sequences from plant pathogen-response (PR) proteins or anti-microbial peptides. In illustrative embodiments, plant anti-microbial peptides comprise amino acid sequences listed in Table 2 and Table 3.

In illustrative embodiments, the peptides comprise amino acid sequences from plant proteins of unknown function. In illustrative embodiments, the peptides comprise amino acid sequences of predicted or hypothetical proteins. In illustrative embodiments, the peptides comprise amino acid sequences listed in Table 4. In illustrative embodiments, the peptides comprise amino acid sequences from host plants that are identical in sequence to regions of peptides from other plant species. In illustrative embodiments, the other plant species comprise Oryza sativa, Hordeum vulgare, Zea mays, Arabidopsis thaliana and Medicago truncatula. In illustrative embodiments, the peptides comprise amino acid sequences listed in Table 5.

The present technology encompasses peptides having amino acids substantially identical to those listed in Tables 1-5. Substantially identical amino acid sequences are those that contain additions, deletions, or substitutions relative to the sequence disclosed herein, but have a high degree of identity to the disclosed sequence when aligned or compared using a sequence comparison algorithm such as BLAST or BLAST 2.0, or when manually aligned and visually inspected. In illustrative embodiments, peptides with substantial identity to those disclosed herein have about 60%, 65%, 70%, 75%, 80%, 85%, 90%, 91%, 92%, 93%, 94%, 95%, 96%, 97%, 98%, 99%, or higher identity over a specified region.

In illustrative embodiments, the present technology comprises an isolated nucleic acid encoding the peptides disclosed herein. In some embodiments, the nucleic acid is a deoxyribonucleic acid (DNA), such as a cDNA or genomic DNA. In some embodiments, the nucleic acid is a ribonucleic acid, such as an mRNA. In some embodiments, the nucleic acid is used to obtain peptide for use in the disclosed methods, for example, as a positive control peptide. In some embodiments, the nucleic acid comprises an expression plasmid for expressing the peptide in a host cell. In some embodiments, the present technology encompasses a host cell comprising a nucleic acid encoding one or more peptides disclosed herein. In illustrative embodiments, the host cell is a prokaryotic cell, a eukaryotic cell, a mammalian cell, or a non-mammalian cell. In some embodiments, the host cell is a bacterial cell, a yeast cell, a plant cell, or an insect cell. In some embodiments, nucleic acids encoding the peptides disclosed herein are expressed in cell-free transcription systems known in the art, such as, for example, a rabbit reticulocyte system or a wheat germ extract system.

Due to the degeneracy of nucleic acid coding sequences, other nucleic acid sequences which encode substantially the same amino acid sequences as those of the naturally occurring proteins may be used in the practice of the present technology. These include, but are not limited to, nucleic acid sequences including all or portions of the nucleic acid sequences encoding the peptides of the present technology, which are altered by the substitution of different codons that encode a functionally equivalent amino acid residue within the sequence, thus producing a silent change. For example, as noted above, one or more amino acid residues within a peptide sequence can be substituted for another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are peptides or fragments or derivatives thereof which are differentially modified such as, for example, by glycosylation, proteolytic cleavage, or linkage to another molecule. Any technique for peptide alteration known in the art can be used, including but not limited to in vitro site directed mutagenesis, J. Biol. Chem. 253:6551 (1978), use of linkers, and the like.

### II. Preparation of the Peptides of the Present Technology

The peptides of the present technology may be synthesized by any of the methods well known in the art. Suitable methods for chemically synthesizing the peptides include, for example, liquid phase and solid phase synthesis, and those methods described by Stuart and Young, Solid Phase Peptide Synthesis, Second Edition, Pierce Chemical Company (1984), and in Methods Enzymol., 289, Academic Press, Inc, New York (1997).

Recombinant peptides may be generated using conventional techniques in molecular biology, protein biochemistry, cell biology, and microbiology, such as those described in Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, NY, 1987); and Meth. Enzymol., Vols. 154 and 155, Wu & Grossman, and Wu, Eds., respectively. Recombinant peptides may be produced through expression of heterologous nucleic acids in host cells, or using cell-free transcription and translation systems known in the art, such as, for example, rabbit reticulocyte lysate of wheat germ agglutinin systems.

Additionally or alternatively, peptides of the present technology may be prepared by isolation from natural sources using methods known in the art, such as, for example, methods described in Wall, et al., Phytopathology 100(9): 848-54 (2010). In illustrative embodiments, peptides described herein are isolated from natural sources comprising starch granules isolated from plants. In illustrative embodiments, the plant comprises a grain. In illustrative embodiments, the plant comprises a non-grain. Illustrative methods for isolating starch granules from grains and non-grains are given in the Examples provided below.

In illustrative embodiments, peptides extracted from the surface of starch granules comprise peptides from plant pathogens. In illustrative embodiments, the presence of such peptides on the surface of a starch granule indicates that the plant has been exposed to a plant pathogen. In illustrative embodiments, the presence of such peptides may indicate an active infection by a plant pathogen or a past infection by a plant pathogen. In illustrative embodiments, the presence of such peptides may indicate exposure to a plant pathogen that did not result in infection of the plant. In illustrative embodiments, the peptides comprise amino acid sequences listed in Table 1.

In illustrative embodiments, peptides extracted from the surface of starch granules comprise peptides from plants. In illustrative embodiments, such peptides comprise peptides of known function or unknown function. In illustrative embodiments, such peptides comprise hypothetical peptides or peptides from plant pathogen-response (PR) proteins. In illustrative embodiments, the peptides are from anti-microbial proteins or proteins related to starch storage and metabolism. In illustrative embodiments, the presence of such peptides on the surface of a starch granule indicates that the plant has been exposed to a plant pathogen. In illustrative embodiments, the presence of such peptides may indicate an active infection by a plant pathogen or a past infection by a plant pathogen. In illustrative embodiments, the presence of such peptides may indicate exposure to a plant pathogen that did not result in infection of the plant. In illustrative embodiments, the peptides comprise amino acid sequences listed in Tables 2-5.

### Methods of the Present Technology

In some aspects, the present technology relates to methods for detecting the exposure of a plant to one ore more plant pathogens. In illustrative embodiments, the plant is a grain. In illustrative embodiments, the grain comprises maize, rice, wheat, barley, sorghum, millet, oats, rye, soybeans, canola, triticale, fonio, buckwheat, quinoa, teff, wild rice, amaranth, kaniwa, spelt, einkorn, emmer, and durum. In illustrative embodiments, the grain comprises the genus Triticum. In illustrative embodiments, the grain comprises Triticum turgidum durum or Triticum aestivum. In some embodiments, the grain comprises maize.

In some aspects, the present technology relates to methods for detecting the exposure of a non-grain to one or more plant pathogens. In illustrative embodiments, the non-grain comprises fruits, nuts, vegetables, legumes, tubers, leaves, trees, flowers, herbs, bushes, grasses, vines, ferns, mosses, fungi, or green algae. In some embodiments, starch rich plant tissues or organs sold in commerce (e.g., cassava, plantain, manioc, cola nuts, coca tree bark, orange peel, orange pulp, bananas, pineapples, apple flesh, pumpkin and squash) are tested.

According to the present methods, exposure of a plant to a plant pathogen is detected by sampling peptides present on the surface of the plant starch granules. In illustrative embodiments, the methods comprise sterilizing or otherwise cleaning starting plant material, physically isolating starch granules from the plant, washing the starch granules to remove residual debris, extracting peptides present at the surface of the starch granule, and identifying peptides present in the extract. Illustrative methods encompassing these steps are given in the Examples provided below.

Starch granules may be isolated from the plant by methods known in the art, such as, for example, those described by Wall, et al., Phytopathology 100(9): 848-54 (2010). Plant samples may be sterilized prior to the isolation of starch granules, in order to prevent contamination of samples with peptide artifacts. Sterilization may be accomplished by methods known in the art, such as, for example, washing the plant material with sodium hypochlorite. Multiple sterilization steps may be employed as needed, depending on the degree to which the starting material requires sterilization. Following sterilization, the plant material may be washed with sterile distilled water or an aqueous buffer, such as, for example, phosphate buffered saline (PBS). Any aqueous buffer or solvent compatible with the methods disclosed herein may be used. The plant material may then be dried prior to additional processing. In illustrative embodiments, wheat seeds are sterilized in 0.25% sodium hypochlorite for 10 minutes, rinsed with distilled water 20-30 times, and dried overnight in a laminar flow hood.

Starch granules may be separated from the plant by methods known it the art, including physical manipulation such as bending, crushing, blending, milling, and grinding. Any method compatible with the methods disclosed herein may be used to disrupt the starting plant material. Starch granules may be isolated from any relevant portion of a plant, including, but not limited to, fruit, seeds, kernels, cobs, husks, rhizomes, tubers, fronds, leaves, roots, petals, flour, anthers, pollen, pollen grains, glumes, awns, straw, palea, pedicels, rachilla, lodicules, stolons, rhizomes, sheaths, collars, blades, auricles, ligules, culms, coffee cherry pulp, coffee ‘bean’ drupes, silverskin, parchment, fruit mucilage, and twigs. The portion of the plant selected for starch isolation will depend on the nature of the plant and the plant pathogens under examination. Isolation of starch granules will require more or less physical manipulation depending on the nature of the starting plant material and the portion of the plant used for isolation. In an illustrative embodiment, wheat seeds are ground into meal using a mortar and pestle or other grinder.

Starch granules may be isolated from the disrupted plant material by passing a solvent through the disrupted plant material and capturing the eluate. In illustrative embodiments, the solvent comprises distilled water or other aqueous buffer. Starch granules may be collected from the eluate by centrifugation. The isolated starch granules may be washed to remove unwanted debris by removing the supernatant from the centrifuged granules, resuspending the granules in sterile distilled water or aqueous buffer, and repeating the centrifugation. The wash step may optionally be repeated as many times as necessary to remove unwanted plant material or other debris. In an illustrative embodiment, 2 g of ground wheat seeds are mixed with distilled water and formed into a dough ball. Starch granules are isolated from the dough ball by passing distilled water around and through the ball and capturing the eluate. In an illustrative embodiment, the starch granules are collected by centrifugation at 1600×g for 2 minutes. In an illustrative embodiment, the isolated starch granules are washed by centrifugation and re-suspension twenty times. In illustrative embodiments, starch granules are isolated through water-washing and decanting. In some embodiments, a starch granule that has a higher density than water (e.g., alpha 1,4/1,6 glucan) may be isolated via water-washing and decanting.

Peptides present on the surface of the starch granule may be extracted by contacting the isolated starch granules with at least one solvent. In illustrative embodiments, the granules are contacted first by an aqueous solvent comprising a protease. In illustrative embodiments, the protease comprises trypsin or chymotrypsin. The granules are then centrifuged and the supernatant removed and retained. In illustrative embodiments, the granules are then washed in distilled water or aqueous buffer by resuspension and centrifugation. In illustrative embodiments, the starch granules are then contacted by an organic solvent. The granules are then centrifuged and the supernatant removed and retained. In illustrative embodiments, the aqueous and organic supernatants are then dried under vacuum and the pellets resuspended in distilled water. In illustrative embodiments, the supernatants are dried at room temperature or greater than room temperature. In illustrative embodiments, the supernatants are dried under vacuum or at atmospheric pressure. Any conditions compatible with the methods described herein may be used to dry the supernatants. In illustrative embodiments, the pellets are then resuspended and purified. The pellets may be resuspended in any buffer compatible with the methods described herein, including distilled water or other aqueous buffer. The resuspended pellets may be purified to remove salt and residual starch using methods known in the art, such as, for example, specialized filtration methods. Specialized filtration products include, but are not limited to, ZipTips® (Millipore, Bedford, Mass., USA).

In illustrative embodiments, 150 mg of water-washed starch granules isolated from wheat seeds is contacted with aqueous buffer comprising 5 μg trypsin in 50 mM sodium bicarbonate, and incubated at 37° C. overnight. In illustrative embodiments, the granules are then centrifuged at 18000×g for 1 minute and the supernatant removed and retained. In illustrative embodiments, the granules are then contacted with 50% isopropanol (v/v) in 50 mM NaCl and mixed at room temperature for 45 minutes. In illustrative embodiments, the starch granules are then centrifuged at 2500×g for 5 minutes and the supernatant removed and retained. In illustrative embodiments, the aqueous and organic supernatants are dried under vacuum at 30° C. for 4 hours, resuspended in 40 μl of distilled water, purified using ZipTips® (Millipore, Bedford, Mass., USA) to remove salt and residual starch, and dried under vacuum at 30° C. for 1 hour.

Peptides extracted from the surface of starch granules may be detected and/or characterized using methods known in the art. Peptides in a sample may be detected, for example, using methods of high performance liquid chromatography (HPLC) such as those described in Aguilar, HPLC of Peptides and Proteins: Methods and Protocols, Humana Press, New Jersey (2004). Peptides may be detected, for example, using reverse-phase HPLC (RP-HPLC) or ion exchange HPLC. In some embodiments, peptides are detected and/or characterized using reverse phase HPLC (RP-HPLC). In some embodiments, peptides are detected and/or characterized using ion-exchange HPLC. In addition, peptides in a sample may be characterized, for example, using methods of mass spectrometry (MS). A general reference related to methods of mass spectrometry is Sparkman, Mass Spectrometry Desk Reference, Pittsburgh: Global View Pub (2000). One of skill in the art will understand that the peptides described herein may be detected and/or characterized using any number of conventional biochemical methods known in the art. The HPLC and MS methods described herein are illustrative and are not to be construed as limiting in any way. Illustrative peptide sequences identified by HPLC and mass spectrometry are given in Example 1.

Peptide identities may be determined by methods known in the art. For example, peptide sequences obtained by mass spectrometry may be compared to a database of peptide sequences, such as, for example, the National Center for Biotechnology Information (NCBI) database maintained by the National Institutes of Health (NIH), Bethesda, Md., USA. In illustrative embodiments, peptide sequences are identified using Mascot (Matrixscience Ltd, Boston, Mass., USA). Illustrative peptide sequences identified using Mascot are given in Example 1.

In illustrative embodiments, peptides extracted from the surface of starch granules comprise peptides from plant pathogens. In illustrative embodiments, the presence of such peptides on the surface of a starch granule indicates that the plant has been exposed to a plant pathogen. In illustrative embodiments, the presence of such peptides may indicate an active infection by a plant pathogen. In illustrative embodiments, the presence of such peptides may indicate a past infection by a plant pathogen. In illustrative embodiments, the presence of such peptides may indicate exposure to a plant pathogen that did not result in infection of the plant. In illustrative embodiments, the peptides comprise amino acid sequences listed in Table 1.

In illustrative embodiments, peptides extracted from the surface of starch granules comprise peptides from plants. In illustrative embodiments, such peptides comprise peptides of known function. In illustrative embodiments, such peptides comprise peptides of unknown function. In illustrative embodiments, such peptides comprise hypothetical peptides or peptides from plant pathogen-response (PR) proteins. In illustrative embodiments, the peptides are from anti-microbial proteins or proteins related to starch storage and metabolism. In illustrative embodiments, the presence of such peptides on the surface of a starch granule indicates that the plant has been exposed to a plant pathogen. In illustrative embodiments, the presence of such peptides is indicative of an active infection by a plant pathogen. In illustrative embodiments, the presence of such peptides is indicative of a past infection by a plant pathogen. In illustrative embodiments, the presence of such peptides is indicative of exposure to a plant pathogen that did not result in infection of the plant. In illustrative embodiments, the peptides comprise one or more amino acid sequences listed in Tables 2-5.

### Kits of the Present Technology

In one aspect, the present technology provides a kit for detecting the exposure of a plant to one or more plant pathogens. In illustrative embodiments, the kit comprises at least one solvent, wherein the at least one solvent extracts peptides present on the surface of the at least one starch granule. In illustrative embodiments, the at least one solvent comprises distilled water or another aqueous solvent. In illustrative embodiments, the aqueous solvent comprises a protease. In illustrative embodiments, the protease comprises trypsin or chymotrypsin.

In illustrative embodiments, the kit comprises at least one negative control starch granule. In illustrative embodiments, the negative control starch granule comprises a starch granule isolated from a pathogen-free plant. In illustrative embodiments, the negative control starch granule comprises a starch granule stripped of surface peptides. In illustrative embodiments, the negative control comprises a starch granule isolated from a plant resistant to Fusarium.

In some embodiments, the kit comprises at least one positive control. In illustrative embodiments, the positive control comprises a starch granule isolated from a pathogen-exposed plant. In illustrative embodiments, the positive control comprises a starch granule isolated from a plant susceptible to Fusarium. In illustrative embodiments, the positive control comprises a peptide from a plant pathogen protein. In illustrative embodiments, the positive control comprises a peptide from a plant protein (e.g., a plant pathogen-response protein). In illustrative embodiments, the positive control peptide is synthetic, recombinant, or isolated from a natural source. In illustrative embodiments, the positive control peptide comprises a peptide listed in Table 1-5.

### Pathogen Detection of the Present Technology

In one aspect, the present technology includes a processing circuit configured to determine the presence of a plant pathogen. A processing circuit includes one or more processors and one or more memory devices. A processor may be a general purpose or specific purpose processor, an application specific integrated circuit (ASIC), a field programmable gate array (FPGA), or another electronic processing component. The one or more processors are communicatively coupled to one or more memory devices and configured to execute computer code or instructions stored in the one or more memory devices. The one or more memory devices of the processing circuit also store data indicative of amino acid sequences (e.g., one or more of the peptide sequences of Tables 1-5, etc.). A memory device may include a RAM, ROM, hard drive storage, non-volatile memory, flash memory, optical memory, non-transitory memory, a remote server, computer readable media (e.g., a CD-ROM, a DVD-ROM, magnetic storage tape, floppy, etc.), or any other suitable memory for storing software objects and/or computer instructions. The processing circuit may additionally include one or more interfaces configured to receive and transmit data (e.g., as input and/or output data).

When the one or more processors execute the instructions stored in the one or more memory devices, the one or more processors may perform a number of operations. The operations include receiving data indicative of a peptide sequence derived from a plant. The operations also include comparing the received data to the amino acid sequence data stored in the one or more memory devices. For example, the stored amino acid sequence data may include amino acid sequences from plant pathogens and non-grain anti-microbial plant proteins. The operations further include providing an indication of the comparison to an electronic device (e.g., a display, a speaker, a computer, a processing circuit, etc.). For example, the indication may be display data provided to an electronic display that causes the display to show visual indicia that the received data includes a peptide sequence indicative of a plant pathogen.

In illustrative embodiments, the peptide data stored in the one or more memories of the processing circuit comprises data indicative of peptides from plant pathogens. In illustrative embodiments, the plant pathogen peptides comprise amino acid sequences listed in Table 1. In illustrative embodiments, the peptides comprise peptides from plants. In illustrative embodiments, the plant peptides comprise peptides listed in Tables 2-5.

In illustrative embodiments, the presence of peptides of the present database on the surface of a plant starch granule (e.g., the received data indicative of a peptide sequence derived from a plant) indicates that the plant has been exposed to a plant pathogen. In illustrative embodiments, the presence of such peptides may indicate an active infection by a plant pathogen. In illustrative embodiments, the presence of such peptides may indicate a past infection by a plant pathogen. In illustrative embodiments, the presence of such peptides may indicate exposure to a plant pathogen that did not result in infection of the plant. In illustrative embodiments, the peptides comprise amino acid sequences listed in Tables 1-5.

The following Examples are presented in order to more fully illustrate the select embodiments of the present technology. These Examples should in no way be construed as limiting the scope of the present technology, as defined by the appended claims.

### EXAMPLES

### Example 1

### Isolation and Identification of Peptides from T. aestivum and T. turgidum durum Starch Granules

Wheat Samples.

Mature seeds of Triticum turgidum durum and Triticum aestivum cultivar AC Andrew were obtained from Eastern Cereal and Oilseed Research Centre (Ottawa, Canada). Reagents were purchased from Sigma-Aldrich (St. Louis, Mo., USA) unless otherwise noted. The preparation of wheat starch granules for mass spectrometric analysis was performed in triplicate.

Starch Granule Extraction.

Wheat seed samples were sterilized in 0.25% sodium hypochlorite (4% domestic bleach solution) for 10 min, rinsed with distilled water 20-30 times, and dried overnight in a laminar flow hood. Seeds were ground into a meal using a separate, clean mortar and pestle for each wheat species. The following procedure was performed in triplicate for each wheat sample: an aliquot of meal (2 g) was mixed with distilled water to form a dough ball. Distilled water was passed through the dough ball and, by gently squeezing the dough ball, a starch granule-rich flow-through was obtained. To collect the granules, this flow-through was centrifuged at 1600×g for 2 minutes; the supernatant was discarded and the starch pellet was resuspended in distilled water. The resuspension and centrifugation wash step was repeated twenty times for each wheat sample.

Sampling the Starch Granule Surface Proteome.

Extraction of peptides from the surface of starch granules was carried out in two phases. First, starch granules were treated with an aqueous solvent (“tryptic shaving”) comprising trypsin in order to remove peptides present on the outer-most surface layer (“distal peptides”). Second, starch granules were contacted with an organic solvent in order to remove more tightly bound, inner layer peptides (“proximal peptides”).

For tryptic shaving, aliquots of water-washed starch granules (approximately 150 mg) were subjected to enzymatic shaving with trypsin (Promega, Madison, Wis.; USA; 5 μg trypsin in 50 mM sodium bicarbonate, 37° C. overnight). Trypsin-treated granules were centrifuged (18000×g, 1 minute) and the aqueous supernatant transferred to a fresh tube. The insoluble starch pellet was washed three times in distilled water. Peptides remaining on the insoluble granules were extracted by suspending the granules in 12 μl 50% isopropanol (v/v), 50 mM NaCl per milligram wet weight. The granule suspensions were mixed at room temperature for 45 minutes and centrifuged at 2500×g for 5 minutes to obtain the isopropanol supernatant. Insoluble pellets were discarded. The aqueous and isopropanol supernatants were both dried under vacuum at 30° C. for 4 h. Peptide pellets were resuspended in 40 μl of distilled water, purified using ZipTips® (Millipore, Bedford, Mass., USA) to remove salt and residual starch, and dried under vacuum at 30° C. for 1 hour. Peptides were resuspended in 40 μl of 0.1% formic acid (v/v), separated by high performance liquid chromatography (HPLC) and analyzed by electrospray ionization mass spectrometry (ESI-MS), as detailed below.

Chromatography and Mass Spectrometry.

Peptides were injected onto a 7 cm×200 μm inner diameter trap column, fitted, and packed in-house with 5 cm of 5 μm YMC ODS A reversed phase packing material (Waters, Milford, Mass.) using an Agilent 1100 HPLC (Agilent, Santa Clara, Calif., USA). The peptides were washed for 4 minutes at 20 μl/min with an aqueous solution containing 5% acetonitrile and 0.1% formic acid (v/v). The trap column was connected in series to a 6 cm×75 μm Picofrit analytical column with a tip opening of 10 μm (New Objective, Woburn, Mass.), packed with 5 cm of YMC ODS A, for mass spectrometric analysis as well as a diverter valve to split the solvent flow prior to the columns. Peptides were eluted from the trap and analytical columns at a flow rate of ˜250 nl/min, ionized by nanoelectrospray ionization (ESI) and analyzed using a QSTAR® Pulsar QqTOF mass spectrometer (Applied Biosystems, Foster City, Calif., USA) operating in information-dependent acquisition mode. Mass analysis included a 1 second survey scan followed by four 3 second tandem mass spectrometric scans on the most intense peaks in the spectrum; masses could be sequenced twice before being added to an exclusion list for 90 seconds. The HPLC pumped 0.1% formic acid (v/v) in water with the following percentage gradient of acetonitrile: 0 min: 5%, 3 min: 15%, 50 min: 40%, 55 min: 50% and 60 min: 80%. MS/MS data were searched against a custom database consisting of all wheat proteins from the comprehensive, non-redundant protein database, NCBInr, using Mascot (Matrixscience Ltd, Boston, Mass., USA). Mass tolerances were set to ±100 ppm and ±0.2 Da for the peptide and fragment ion spectra, respectively; up to two missed cleavages were considered. Oxidation of methionine residues was selected as a variable modification; no fixed modifications were selected as the ‘tryptic shaving’ did not involve an alkylation step. Mass spectral significance was determined by searching the data against a “decoy” database containing a copy of the NCBInr database in reverse order and adjusting the ion score cutoff to ≧15 such that the false positive rate (proportional to the number of peptides matching to reversed sequences in the database) was equal to or less than 1%. Each match from the database was verified manually; data were deemed acceptable if at least three successive y- or b-ions were present (or y-++ or b-++ ions if the charge state of the peptide was greater than 2+).

Results.

All four peptide samples (aqueous supernatant and isopropanol supernatant for both the hard and soft wheat) were analyzed by mass spectrometry.

The results of the mass spectrometry analysis and Mascot protein identification are presented in five tables based on several criteria discussed below. Compiling fragment ion spectra from all four peptide samples, Table 1 lists all peptides identical to regions of several known plant pathogen proteins, as annotated in the NCBI protein database.

TABLE 1
List of peptides identical to regions of proteins from the plant pathogens Agrobacterium
tumefaciens, Fusarium graminearum, Pectobacterium carotovora, Magnaporthe grisea, Xanthomonas
axonopodis and X. oryzae, generated from mass spectrometric analysis of aqueous supernatants
and isopropanol extracts of trypsin-treated hard and soft wheat starch granules.

| | | |
|---|---|---|
| gi\|50123425 | Bifunctional N- | (K)LDDPAGYGRIVR(E) |
| | acetylglucosamine-1- | (SEQ ID NO: 1) |
| | phosphate | |
| | uridyltransferase | |
| | [E. carotovora] | |
| gi\|39941602 | Hypothetical protein | (R)DRVADAWPVFCK(Y) |
| | MG5712.4 | (SEQ ID NO: 2) |
| | [M. grisea] | |
| gi\|46121515 | Hypothetical protein | (R)ALGKSKNAR(T) |
| | FG05136.1 | (SEQ ID NO: 3) |
| | [F. graminearum] | |
| gi\|84623867 | Hypothetical protein | (R)ATLANRKR(V) |
| | XOO_2210 | (SEQ ID NO: 4) |
| | [X. oryzae] | |
| gi\|39977703 | Hypothetical protein | (R)VRAEMVAKMDEK(G) |
| | MG06736.4 | (SEQ ID NO: 5) |
| | [M. grisea] | |
| gi\|50121895 | Putative branched-chain | (K)VVDTAAKKR(G) |
| | amino acid | (SEQ ID NO: 6) |
| | aminotransferase | |
| | [E. carotovora] | |
| gi\|39942636 | Hypothetical protein | (K)ASRVRDSQRASR(A) |
| | MG03398.4 | (SEQ ID NO: 7) |
| | [M. grisea] | |
| gi\|39940010 | Hypothetical protein | (K)IAMSELAR(R) |
| | MG05235.4 | (SEQ ID NO: 8) |
| | [M. grisea] | |
| gi\|15891657 | Hypothetical protein | (K)ACPTAEMRGVALGR(F) |
| | AGR_L_3085 | (SEQ ID NO: 9) |
| | [A. tumefaciens] | |
| gi\|15891318 | Hypothetical protein | (K)KPTAQEIAITR(G) |
| | AGR_L_2410 | (SEQ ID NO: 10) |
| | [A. tumefaciens] | |
| gi\|15891555 | Hypothetical protein | (R)VVVMQDGR(I) |
| | AGR_L_2889 | (SEQ ID NO: 11) |
| | [A. tumefaciens] | |
| gi\|16119407 | Hypothetical protein | (R)LSITPAAVSRNVAMLER(N) |
| | AGR_pAT_256 | (SEQ ID NO: 12) |
| | [A. tumefaciens] | |
| gi\|21243478 | D-amino acid | (R)AREVVIATGPWSPALAAQLG |
| | dehydrogenase subunit | LR(L) |
| | [X. axonopodis] | (SEQ ID NO: 13) |
| gi\|21242862 | Hypothetical protein | (R)MRKTLLAR(H) |
| | XAC2127 | (SEQ ID NO: 14) |
| | [X. axonopodis] | |
| gi\|21245076 | Oxidoreductase | (M)NIPVPVVR(L) |
| | [X. axonopodis] | (SEQ ID NO: 15) |

^{a}The reference column provides the GenBank GI number for the most likely protein candidate for which peptides were identified.
^{b}Where peptides were detected as having an oxidized methionine, these residues have been underlined.

To assess the nature of the starch granule surface proteome that may be either distantly tethered or, conversely, tightly bound, the aqueous tryptic supernatants are presented separately in Tables 2 and 3, respectively. Table 2 lists peptides based upon two criteria: (a) those peptides found distal to the starch granule surface, and thus released into the aqueous supernatant following tryptic digestion, and (b) reported as being antimicrobial, as annotated in the NCBI protein database. Table 3, similar to Table 2, lists peptides annotated by the NCBI protein database as antimicrobial in function, but is restricted to peptides found proximal to the starch granule surface. Proximal peptides are defined as those peptides which remain bound to the starch granule surface following tryptic digestion and collected using 50% (v/v) isopropanol, 50 mM NaCl.

TABLE 2
List of peptides identical to regions of known antimicrobial proteins, as annotated by the
NCBI database, generated from mass spectrometric analysis of aqueous supernatants of trypsin-
treated hard and soft wheat starch granules.

| | | Peptide sequence^{b} | |
|---|---|---|---|
| Reference^{a} | Protein | Hard wheat | Soft wheat |
| gi\|391929 | Tritin | (K)LTNVALGR(Q) | (R)DLLGDTDKLTNVAL |
| | [T. aestivum] | (SEQ ID NO: 16) | GR(Q) (SEQ ID NO: 32) |
| | | (R)ADNLYWEGFK(S) | |
| | | (SEQ ID NO: 17) | |
| | | (K)TSPASTGLTLATR(A) | |
| | | (SEQ ID NO: 18) | |
| | | (R)FQTVSGFVAGVLEPK(E) | |
| | | (SEQ ID NO: 19) | |
| | | (K)AQVNGWQDLSEALLK(T) | |
| | | (SEQ ID NO: 20) | |
| | | (R)EAVTTLLLMVHEATR(F) | |
| | | (SEQ ID NO: 21) | |
| | | (R)DLLGDTDKLTNVALGR(Q) | |
| | | (SEQ ID NO: 22) | |
| | | (K)SSDGTWWELTPGLIPG | |
| | | ATHVGFGGTYR(D) | |
| | | (SEQ ID NO: 23) | |
| gi\|5669008 | Secretory protein | (K)VLSDASAFIWK(T) | (K)SGYTDDFFAQILGK(N) |
| | [T. aestivum] | (SEQ ID NO: 24) | (SEQ ID NO: 33) |
| | | (K)SGYTDDFFAQILGK(N) | |
| | | (SEQ ID NO: 25) | |
| gi\|62465514 | Class II chitinase | (R)AIGVDLLSNPDLVATD | (R)AIGVDLLSNPDLVAT |
| | [T. aestivum] | PTVSFK(T) | DPTVSFK(T) |
| | | (SEQ ID NO: 26) | (SEQ ID NO: 34) |
| gi\|22001285 | Peroxidase 1 | (K)DIGLAAGLLR(L) | (K)DIGLAAGLLR(L) |
| | [T. aestivum] | (SEQ ID NO: 27) | (SEQ ID NO: 35) |
| | | (R)DSVVVSGGPDYR(V) | (R)EGLFVSDQDLFTNDI |
| | | (SEQ ID NO: 28) | TRPIVER(F) |
| | | (R)EGLFVSDQDLFTNDITR | (SEQ ID NO: 36) |
| | | PIVER(F) | |
| | | (SEQ ID NO: 29) | |
| gi\|132577 | Protein synthesis inhibitor | (K)LTNVALGR(Q) | |
| | I (Ribosome-inactivating | (SEQ ID NO: 16) | |
| | protein I) | (R)FQTVSGFVAGLLHPK(A) | |
| | [H. vulgare] | (SEQ ID NO: 30) | |
| | | (R)DLLGDTDKLTNVALGR(Q) | |
| | | (SEQ ID NO: 22) | |
| gi\|55669876 | Chain X, Crystal structure | (-)LPVLAPVTK(D) | |
| | of xylanase inhibitor I | (SEQ ID NO: 31) | |
| | [T. aestivum] | | |
| gi\|20804336 | Xylanase inhibitor protein | | (R)ALATGIFER(A) |
| | I | | (SEQ ID NO: 37) |
| | [T. aestivum] | | |
| gi\|116344 | Endochitinase precursor | | (K)SAQKTER(F) |
| | (MF1 antigen) | | (SEQ ID NO: 38) |
| | [Brugia malayi] | | |
| gi\|2894149 | Monomeric alpha- | | (K)VPIPNPSGDR(A) |
| | amylase inhibitor | | (SEQ ID NO: 39) |
| | [T. aestivum] | | |
| gi\|1709917 | Puroindoline-B precursor | | (K)DFPVTWPTK(W) |
| | [T. aestivum] | | (SEQ ID NO: 40) |

^{a}The reference column provides the GenBank GI number for the most likely protein candidate for which peptides were identified.
^{b}Amino acids in parentheses indicate residues expected to flank each identified peptide as determined from the corresponding GenBank protein's amino acid sequence. N-terminal peptides are indicated by (-) at the N-terminus.

TABLE 3
List of peptides identical to regions of known wheat antimicrobial proteins, generated from
mass spectrometric analysis of isopropanol extracts of trypsin-treated hard and soft wheat
starch granules.

| | | Peptide sequence^{b} | |
|---|---|---|---|
| Reference^{a} | Protein | Hard wheat | Soft wheat |
| gi\|391929 | Tritin | (R)ADNLYWEGFK(S) | (K)TSPASTGLTLATR(A) |
| | [T. aestivum] | (SEQ ID NO: 17) | (SEQ ID NO: 44) |
| | | (K)TSPASTGLTLATR(A) | (R)QQMADAVTALYGR(T) |
| | | (SEQ ID NO: 18) | (SEQ ID NO: 45) |
| | | (R)QQMADAVTALYGR(T) | (K)AQVNGWQDLSEALLK(T) |
| | | (SEQ ID NO: 41) | (SEQ ID NO: 46) |
| | | (R)FQTVSGFVAGVLHPK(E) | (R)DLLGDTDKLTNVAL |
| | | (SEQ ID NO: 42) | GR(Q) (SEQ ID NO: 32) |
| | | (K)AQVNGWQDLSEALLK(T) | |
| | | (SEQ ID NO: 20) | |
| | | (R)EAVTTLLLMVHEATR(F) | |
| | | (SEQ ID NO: 21) | |
| | | (R)DLLGDTDKLTNVALG | |
| | | R(Q) (SEQ ID NO: 22) | |
| | | (R)TAEQAAATLGILLFVQ | |
| | | VPGGMTVAQALELFHK(S) | |
| | | (SEQ ID NO: 43) | |
| gi\|5669008 | Secretory protein | (K)VLSDASAFIWK(T) | (K)SGYTDDFFAQILGK(N) |
| | [T. aestivum] | (SEQ ID NO: 24) | (SEQ ID NO: 33) |
| | | (K)SGYTDDFFAQILGK(N) | |
| | | (SEQ ID NO: 25) | |
| gi\|62465514 | Class II chitinase | (R)AIGVDLLSNPDLVATD | (R)GPIQLSHNYNYGPAG |
| | [T. aestivum] | PTVSFK(T) | R(A) (SEQ ID NO: 47) |
| | | (SEQ ID NO: 26) | (R)AIGVDLLSNPDLVAT |
| | | | DPTVSFK(T) |
| | | | (SEQ ID NO: 34) |
| gi\|22001285 | Peroxidase 1 | (K)DIGLAAGLLR(L) | |
| | [T. aestivum] | (SEQ ID NO: 27) | |
| gi\|20804336 | Xylanase inhibitor | | (R)ALATGIFER(A) |
| | protein I | | (SEQ ID NO: 37) |
| | [T. aestivum] | | |

^{a}Amino acids in parentheses indicate residues expected to flank each identified peptide as determined from the corresponding GenBank protein's amino acid sequence.
^{b}The reference column provides the GenBank GI number for the most likely protein candidate for which peptides were identified.

To highlight the fact that there were, from all four samples, peptides identical to regions of hypothetical or uncharacterized proteins, or proteins of unknown function, such peptides have been compiled into Table 4. Peptides from all four samples identical to regions of plant proteins which are not antimicrobial are listed in Table 5.

TABLE 4
List of peptides identical to regions of proteins identified by Mascot as being hypothetical
or of unknown function, generated from mass spectrometric analysis of aqueous supernatants
and isopropanol extracts of trypsin-treated hard and soft wheat starch granules.

| | | Peptide sequenc^{b,c} | |
|---|---|---|---|
| Reference^{a} | Protein | Hard wheat | Soft wheat |
| gi\|1323750 | Unknown protein | (R)ANGGLIEGIADYVR(L) | (R)ANGGLIEGIADYVR(L) |
| | [T. aestivum] | (SEQ ID NO: 48) | (SEQ ID NO: 63) |
| gi\|22238 | Unnamed protein | (R)VPTVDVSVVDLTVR(I) | |
| | product | (SEQ ID NO: 49) | |
| | [Z. mays] | | |
| gi\|14662644 | Unknown protein [A. | (R)VLIVVNTEER(K) | |
| | thaliana] | (SEQ ID NO: 50) | |
| gi\|92874420 | At1g27150/T7N9_21- | (M)LAFPLLELGQMK(E) | |
| | related[M. truncatula] | (SEQ ID NO: 51) | |
| gi\|38345325 | OSJNBa0004N05.22 | (R)NTAGEEKQQTLALR(V) | |
| | [O. sativa] | (SEQ ID NO: 52) | |
| gi\|115485083 | Os11g0276000 | (K)SCLMPLRK(S) | |
| | [O. sativa] | (SEQ ID NO: 53) | |
| gi\|115440299 | Os01g0778800 | (K)FDRVESEVALLR(E) | |
| | [O. sativa] | (SEQ ID NO: 54) | |
| gi\|125572766 | Hypothetical protein | (R)SAKPQHR(I) | |
| | OsJ_004106 | (SEQ ID NO: 55) | |
| | [O. sativa] | | |
| gi\|79561588 | Unknown protein | (M)VEMMRSENHLR(Q) | |
| | [A. thaliana] | (SEQ ID NO: 56) | |
| gi\|50428631 | Protein of unknown | (K) CDNMASQYFVCCR(W) | |
| | fuction [O. sativa] | (SEQ ID NO: 57) | |
| gi\|12321294 | Unknown protein | (K)TPPPPPPPPPPLPSR(S) | |
| | [A. thaliana] | (SEQ ID NO: 58) | |
| gi\|18087666 | Unknown protein | (R)IVVADPPYLSK(E) | |
| | [O. sativa] | (SEQ ID NO: 59) | |
| gi\|15223729 | Unknown protein | (K)QVEEMVLVKWVEWKK(E) | |
| | [A. thaliana] | (SEQ ID NO: 60) | |
| gi\|51090857 | Hypothetical protein | (R)IPRWRRR(L) | |
| | [O. sativa] | (SEQ ID NO: 61) | |
| gi\|115434166 | Os01g0116100 | (K)CSIAKDKPKR(N) | |
| | [O. sativa] | (SEQ ID NO: 62) | |
| gi\|18972 | Unnamed protein | | (R)AIGVDLLR(N) |
| | product | | (SEQ ID NO: 64) |
| | [H. vulgare] | | (R)NPDLVATDPTVSFK(T) |
| | | | (SEQ ID NO: 65) |
| | | | (R)GPIQLSHNYNYGPAGR(A) |
| | | | (SEQ ID NO: 47) |
| gi\|37718800 | Hypothetical protein | | (R)SQAKAVAPTSRAR(S) |
| | [O. sativa] | | (SEQ ID NO: 66) |
| gi\|115434624 | Os01g0157200 | | (R)EAASLAEAAK(D) |
| | [O. sativa] | | (SEQ ID NO: 67) |
| gi\|15225665 | Unknown protein | | (R)VVESMRSK(S) |
| | [A. thaliana] | | (SEQ ID NO: 68) |
| gi\|32400760 | Unknown protein | | (R)QLVQIPEQAR(C) |
| | [T. aestivum] | | (SEQ ID NO: 69) |
| gi\|38346495 | OSJNBa0020102.9 | | (R)CSAKYLK(S) |
| | [O. sativa] | | (SEQ ID NO: 70) |
| gi\|24414171 | Hypothetical protein | | (K)VVTVSARPK(-) |
| | [O. sativa] | | (SEQ ID NO: 71) |
| gi\|9755378 | F17F8.11 | | (R)ASKSGLHR(V) |
| | [A. thaliana] | | (SEQ ID NO: 72) |
| gi\|125533309 | Hypothetical protein | | (R)VQKASISLVMK(L) |
| | OsI_033816 | | (SEQ ID NO: 73) |
| | [O. sativa] | | |
| gi\|6692258 | Unknown protein | | (K)NPLAIVNVKGR(A) |
| | [A. thaliana] | | (SEQ ID NO: 74) |

^{a}The reference column provides the GenBank GI number for the most likely protein candidate for which peptides were identified.
^{b}Amino acids in parentheses indicate residues expected to flank each identified peptide as determined from the corresponding GenBank protein's amino acid sequence. C-terminal peptides are indicated by (-) at the C-terminus.
^{c}Where peptides were detected as having an oxidized methionine, these residues have been underlined.

TABLE 5
List of peptides identical to regions of proteins from the plant species Oryza sativa,
Hordeum vulgare, Zea mays, Arabidopsis thaliana and Medicago truncatula, generated from mass
spectrometric analysis of aqueous supernatants and isopropanol extracts of trypsin-treated
hard and soft wheat starch granules.

| | | Peptide sequence^{b,c} | |
|---|---|---|---|
| Reference^{a} | Protein | Hard wheat | Soft wheat |
| gi\|4760582 | Starch synthase (GBSSI) | (K)DAWDTSVVSEIK(V) | |
| | [T. aestivum] | (SEQ ID NO: 75) | |
| | | (K)EALQAEVGLPVDR(K) | |
| | | (SEQ ID NO: 76) | |
| | | (K)FLAANYDVTTALEGK(A) | |
| | | (SEQ ID NO: 77) | |
| | | (K)SSFDFIDGYDKPVEGR(K) | |
| | | (SEQ ID NO: 78) | |
| | | (K)VLTVSPYYAEELISGE | |
| | | AR(G) (SEQ ID NO: 79) | |
| gi\|32400764 | Beta amylase | (R)YDPTAYNTILR(N) | |
| | [T. aestivum] | (SEQ ID NO: 80) | |
| | | (R)FFVDNGTYLTEQGR(F) | |
| | | (SEQ ID NO: 81) | |
| | | (K)AAAAMVGHPEWEFPR | |
| | | (D) (SEQ ID NO: 82) | |
| gi\|169777 | Beta amylase | (K)SAPEELVQQVLSAGW | |
| | [O. sativa] | R(E) (SEQ ID NO: 83) | |
| | | (R)NIEYLTLGVDDQPLFH | |
| | | GR(T) (SEQ ID NO: 84) | |
| gi\|1323750 | Unknown protein | (R)ANGGLIEGIADYVR(L) | (R)ANGGLIEGIADYVR(L) |
| | [T. aestivum] | (SEQ ID NO: 48) | (SEQ ID NO: 63) |
| gi\|167004 | Embryo globulin | (R)VAIMEVNPR(A) | |
| | [H. vulgare] | (SEQ ID NO: 85) | |
| | | (R)VAIMEVNPR(A) | |
| | | (SEQ ID NO: 86) | |
| | | (R)DTFNLLEQRPK(I) | |
| | | (SEQ ID NO: 87) | |
| gi\|228310 | Globulin 2 | (K)EGEGVIVLLR(G) | (K)EGEGVIVLLR(G) |
| | [Z. mays] | (SEQ ID NO: 88) | (SEQ ID NO: 100) |
| gi\|17425168 | Low-molecular weight | (R)TTTRVPFGVGTGVGG | |
| | glutenin subunit group 3 | Y(-) (SEQ ID NO: 89) | |
| | type II | | |
| | [T. aestivum] | | |
| gi\|15220239 | RNA binding protein | (K)IVAILGSMRVPN(-) | |
| | [A. thaliana] | (SEQ ID NO: 90) | |
| gi\|15148385 | Gamma-gliadin | (K)APFASIVADIGGQ(-) | |
| | [T. aestivum] | (SEQ ID NO: 91) | |
| gi\|607198 | 15 kDa grain softness | (K)AIWTSIQGDLSGFK(G) | |
| | protein | (SEQ ID NO: 92) | |
| | [T. aestivum] | | |
| gi\|22238 | Unnamed protein | (R)VPTVDVSVVDLTVR(I) | |
| | product | (SEQ ID NO: 49) | |
| | [Z. mays] | | |
| gi\|15241221 | Structural molecule | (K)VLRSRSRR(L) | |
| | [A. thaliana] | (SEQ ID NO: 93) | |
| gi\|4662644 | Unknown protein | (R)VLIVVNTEER(K) | |
| | [A. thaliana] | (SEQ ID NO: 50) | |
| gi\|12323783 | Putative exportin, tRNA | (R)SKVTSFIHR(M) | |
| | [A. thaliana] | (SEQ ID NO: 94) | |
| gi\|92874420 | At1g27150/T7N9_21- | (M)LAFPLLELGQMK(E) | |
| | related | (SEQ ID NO: 51) | |
| | [M. truncatula] | | |
| gi\|886965 | Low molecular weight | (R)QLPQIPEQSR(Y) | |
| | glutenin | (SEQ ID NO: 95) | |
| | [T. aestivum] | | |
| gi\|38345325 | OSJNBa0004N05.22 | (R)NTAGEEKQQTLALR(V) | |
| | [O. sativa] | (SEQ ID NO: 52) | |
| gi\|115485083 | Os11g0276000 | (K)SCLMPLRK(S) | |
| | [O. sativa] | (SEQ ID NO: 53) | |
| gi\|115440299 | Os01g0778800 | (K)FDRVESEVALLR(E) | |
| | O. sativa] | (SEQ ID NO: 54) | |
| gi\|125572766 | Hypothetical protein | (R)SAKPQHR(I) | |
| | OsJ_004106 | (SEQ ID NO: 55) | |
| | [O. sativa] | | |
| gi\|79561588 | Unknown protein | (M)VEMMRSENHLR(Q) | |
| | [A. thaliana] | (SEQ ID NO: 56) | |
| gi\|50428631 | Protein of unknown | (K)CDNMASQYFVCCR(W) | |
| | function | (SEQ ID NO: 57) | |
| | [O. sativa] | | |
| gi\|12321294 | Unknown protein | (K)TPPPPPPPPPPLPSR(S) | |
| | [A. thaliana] | (SEQ ID NO: 58) | |
| gi\|18401374 | Transcription factor | (K)TLWSRAILSKAVK(L) | |
| | [A. thaliana] | (SEQ ID NO: 96) | |
| gi\|18087666 | Unknown protein | (R)IVVADPPYLSK(E) | |
| | [O. sativa] | (SEQ ID NO: 59) | |
| gi\|15223729 | Unknown protein | (K)QVEEMVLVKWVEWK | |
| | [A. thaliana] | K(E) (SEQ ID NO: 60) | |
| gi\|51090857 | Hypothetical protein | (R)IPRWRRR(L) | |
| | [O. sativa] | (SEQ ID NO: 61) | |
| gi\|15232073 | Lyase/pectate lyase | (R)IYVVTSPR(D) | |
| | [A. thaliana] | (SEQ ID NO: 97) | |
| gi\|115434166 | Os01g0116100 | (K)CSIAKDKPKR(N) | |
| | [O. sativa] | (SEQ ID NO: 62) | |
| gi\|75282488 | Protein H2A.5 | (R)LLAGVTIAHGGVIPNI | |
| | [T. aestivum] | NSVLLPK(K) | |
| | | (SEQ ID NO: 98) | |
| gi\|871551 | Serpin (protease | (K)ISFGIEASDLLK(C) | |
| | inhibitor) | (SEQ ID NO: 99) | |
| | [T. aestivum] | | |
| gi\|18972 | Unnamed protein | | (R)AIGVDLLR(N) |
| | product | | (SEQ ID NO: 64) |
| | [H. vulgare] | | (R)NPDLVATDPTVSFK(T) |
| | | | (SEQ ID NO: 65) |
| | | | (R)GPIQLSHNYNYGPAG |
| | | | R(A) (SEQ ID NO: 47) |
| gi\|37718800 | Hypothetical protein | | (R)SQAKAVAPTSRAR(S) |
| | [O. sativa] | | (SEQ ID NO: 66) |
| gi\|92875830 | Trp repressor/replication | | (K)VFSDEVIR(F) |
| | initiator | | (SEQ ID NO: 101) |
| | [M. truncatula] | | |
| gi\|92893314 | Ribonuclease H; | | (K)KIWSKDIPPSK(S) |
| | Glutathione S- | | (SEQ ID NO: 102) |
| | transferase, C-terminal- | | |
| | like | | |
| | [M. truncatula] | | |
| gi\|115434624 | Os01g0157200 | | (R)EAASLAEAAK(D) |
| | [O. sativa] | | (SEQ ID NO: 67) |
| gi\|15225665 | Unknown protein | | (R)VVESMRSK(S) |
| | [A. thaliana] | | (SEQ ID NO: 68) |
| gi\|32400760 | Unknown protein | | (R)QLVQIPEQAR(C) |
| | [T. aestivum] | | (SEQ ID NO: 69) |
| gi\|54291437 | Prl-like protein | | (R)RELESGKEEGR(R) |
| | [O. sativa] | | (SEQ ID NO: 103) |
| gi\|14587294 | Putative permease 1 | | (R)GMPWWVPFR(S) |
| | [O. sativa] | | (SEQ ID NO: 104) |
| | | | (R)GMPWWVPFR(S) |
| | | | (SEQ ID NO: 105) |
| gi\|38346495 | OSJNBa0020IO2.9 | | (R)CSAKYLK(S) |
| | [O. sativa] | | (SEQ ID NO: 70) |
| gi\|24414171 | Hypothetical protein | | (K)VVTVSARPK(-) |
| | [O. sativa] | | (SEQ ID NO: 71) |
| gi\|9755378 | F17F8.11 | | (R)ASKSGLHR(V) |
| | [A. thaliana] | | (SEQ ID NO: 72) |
| gi\|125533309 | Hypothetical protein | | (R)VQKASISLVMK(L) |
| | OsI_033816 | | (SEQ ID NO: 73) |
| | [O. sativa] | | |
| gi\|50726319 | Putative BRCA1 | | (K)DPVPVNTRRSK(F) |
| | interacting protein C- | | (SEQ ID NO: 106) |
| | terminal helicase 1 | | |
| | [O. sativa] | | |
| gi\|6692258 | Unknown protein | | (K)NPLAIVNVKGR(A) |
| | [A. thaliana] | | (SEQ ID NO: 74) |
| gi\|15229913 | CYP71B38; heme | | (K)KVPLVLIQTSHR(W) |
| | binding/iron ion | | (SEQ ID NO: 107) |
| | binding/monooxygenase/ | | |
| | oxygen binding | | |
| | [A. thaliana] | | |
| gi\|30688003 | Phosphoinositide 5- | | (R)NFKLTLIAR(R) |
| | phosphatase | | (SEQ ID NO: 108) |
| | [A. thaliana] | | |

^{a}The reference column provides the GenBank GI number for the most likely protein candidate for which peptides were identified.
^{b}Amino acids in parentheses indicate residues expected to flank each identified peptide as determined from the corresponding GenBank protein's amino acid sequence. C-terminal peptides are indicated by (-) at the C-terminus.
^{c}Where peptides were detected as having an oxidized methionine, these residues have been underlined.

The Proteome of the Starch Granule Surface.

Several peptides identical to regions of proteins from common plant pathogens were identified on the surfaces of starch granules (Table 1). The species to which peptide identifications were made include F. graminearum, P. carotovora, X. oryzae, Agrobacterium tumefaciens, Magnaporthe grisea, and Xanthomonas axonopodis. The hard wheat contained peptides identical to regions of proteins from all the above mentioned species, while the soft wheat only contained peptides identical to regions of proteins of the plant pathogen X. axonopodis. The majority of the proteins listed in Table 1 are hypothetical proteins derived from genomic ORF sequences in the NCBI nucleotide database. However, several proteins listed in Table 1 have been previously characterized and are known to localize to the cytosol, as annotated in the NCBI protein database. For example, one 14-mer peptide was identical to a region of a bifunctional N-acetylglucosamine-1-phosphate uridyltransferase, an enzyme involved in bacterial cell wall synthesis (gi|50123425).

Among the peptides identical to regions of plant proteins, several proteins belong to classes that have antimicrobial activity, as annotated in the NCBI protein database. These proteins are organized into Table 2 (aqueous supernatant distal peptides) and Table 3 (isopropanol supernatant proximal peptides). Seven unique antimicrobial proteins were identified in the aqueous supernatant of trypsin-treated hard wheat starch granules as opposed to nine in the aqueous supernatant of trypsin-treated soft wheat starch granules (Table 2). While most were identified by a single peptide, a few peptides corresponded to regions of the same protein. For example, ten peptides, each identical to a region of the ribosome-inactivating protein tritin (gi|392929), were found in both the aqueous supernatant (Table 2) and isopropanol supernatant (Table 3) of the hard wheat sample. From soft wheat, only one peptide of tritin was found in the aqueous supernatant whereas four peptides of tritin were found in the isopropanol supernatant.

From all four samples, several peptides were identical to regions of hypothetical plant proteins (genomic ORF only) or plant proteins of unknown function (protein sequence only). These peptides have been compiled into Table 4 and represent a source of novel starch granule-associated proteins.

Peptides from all four samples that were found to match regions of plant proteins are listed in Table 5. They comprise a variety of protein classes, including starch granule synthesis (gi|4760582 Starch synthase, GBSSI), storage (gi|228310 Globulin 2) and transcription (gi|18401374 Transcription factor). Proteins such as GBSSI and grain softness protein (gi|607198) are known to be strongly associated with the starch granule surface. Overall, more peptides were identified in the hard wheat (52 peptides) versus the soft wheat samples (30 peptides). Finally, several peptides corresponded to proteins of related organisms with no homologous proteins sequenced in wheat as well as numerous peptides that have not been sequenced in any organism.

Proteins of Pathogen Origin.

Peptides identical to regions of proteins of plant pathogens were identified at the starch granule surface (Table 1). More peptides (thirteen peptides from six pathogen species) were identified from hard wheat starch granules than from soft wheat starch granules (two peptides from one pathogen species). Pathogen proteins closely associated with the starch granules of wheat may be the molecular remnants of a failed pathogen attack against the developing or mature granules of the wheat kernel. It has been shown in the art that a GFP-expressing Fusarium species is able to infect the majority of a wheat seed coat 16 days after inoculation, though no infection of the endosperm was observed using this method. However, microscopy of F. graminearum-infected wheat and barley endosperm have shown pitted starch granules and fungal hyphae. Therefore, it is not surprising to find remnant proteins of fungal pathogens within the endosperm of mature wheat kernels that serve as molecular ‘footprints’ of plant pathogen exposure. Such molecular footprints of grain shipments may be used to trace the cumulative historical record of the grain's incurred biotic stresses or pathogen load.

Of the pathogen proteins identified (Table 1), several are cytosolic and serve no known direct role in pathogenicity. These include an oxidoreductase (gi|245076), a D-amino acid dehydrogenase subunit (gi|243478), a putative branched-chain amino acid aminotransferase (gi|50121895) and a bifunctional N-acetylglucosamine-1-phosphate uridyltransferase (gi|50123425). This suggests that bacterial pathogens and fungal hyphae were lysed during seed maturation and fixed in place within the desiccated endosperm.

The presence of peptides identical to regions of plant pathogen proteins suggests that the tryptic shaving and mass spectrometric approach of probing the starch granule surface goes beyond providing insight into the starch granule proteome only: it provides a view of the interaction between plant and pathogen.

Host Defense Proteins.

Defense-related proteins are known to comprise an important fraction of the plant proteome. Peptides identical to regions of several antimicrobial proteins, as annotated in the NCBI protein database, were identified in the supernatant and granule-bound isolates of tryptically shaved hard and soft wheat starch granules (Tables 2 and 3). When present at the starch granule surface, these proteins may play a role in protecting the starch granule from invading pathogens. This would ensure that the starch granules are preserved exclusively for the developing plant embryo. A peptide identical to a region of an amylase inhibitor was identified in the supernatant (gi|2894148; Table 2). Without wishing to be bound by theory, alpha amylase and other glycosylase inhibitors may play a role in preventing the breakdown of starch and cell wall components by invading pathogens or may play a role in regulating growth and degradation of the starch granule by the plant itself. A xylanase inhibitor (gi|20804336; Tables 1, 2 and 3), specifically a XIP (xylanase-inhibiting protein)-type inhibitor associated with the starch granules of soft wheat was also detected. A proteomic analysis by others of four commercial starches identified xylanase inhibitors. This result supports the assertion that such enzymes may play a role in antimicrobial activity. XIP is exclusively expressed in wheat endosperm and has been suggested to play a role in plant defense. Although XIP-type xylanase inhibitors are secreted into the intercellular matrix, it may be possible that during seed maturation and desiccation some of the highly-expressed xylanase inhibitors become associated with the starch granule surface.

Hypothetical Proteins.

The proteome of any organism is highly diverse and the identification and characterization of most proteomes is largely incomplete. The motivation to characterize novel proteins generally depends on their candidacy for connection or interaction with a characterized system. By limiting our analysis to the starch granule proteome, novel identifications may be related to starch granule defense or metabolism.

The use of mass spectrometric techniques to discover unknown and hypothetical proteins localized to specific organellar compartments, such as the starch granule surface, may aid in expanding the body of knowledge of host-pathogen interaction and perhaps result in the discovery of novel resistance mechanisms. For example, the unknown protein with GenBank GenInfo Identifier (GI) gi|1323750 (Table 4) is the protein product of a cDNA sequence (gi|1323750). This cDNA sequence from wheat was entered into the database in 1996 and contains an open reading frame annotated as “unknown.” Subsequently, this protein of unknown function was found to match the superfamily of basic secretory protein (BSP) class of proteins thought to play a role in host defense. Other hypothetical proteins, such as OsJ_{—}004106 (gi|125572766), did not match with any known proteins nor do they contain any known conserved domains when analyzed using the NCBI BLAST protein search tools. These hypothetical proteins can therefore no longer be considered hypothetical as the present peptide extraction actually recovered them from granules as bona fide plant proteins.

Host Proteins Related to Storage and Metabolism.

Similar to other proteomic studies of wheat and potato amyloplasts, a number of proteins related to defense (Table 2 and Table 3) and starch granule metabolism and storage (Table 5) were identified. These include the starch metabolism enzymes granule-bound starch synthase I (GBSS I) and beta amylase, as well as the storage protein globulin, identified here as the barley protein, embryo globulin. The identification of proteins already known to associate with the starch granule, such as GBSS I and grain softness protein, validates the efficacy of this current ‘tryptic shaving’ technique to probe the starch granule surface proteome and extend this organellar approach to the investigation of host-pathogen interactions. Peptides identical to regions of proteins from the cereals rice, barley and corn as well as the model plants Arabidopsis and alfalfa, were identified. For these protein hits, it is most likely that the reason they are annotated as non-wheat is that they share sequence identity with homologs from wheat which have not yet had DNA, RNA or protein sequence information reported and are therefore only annotated under related organisms which have had their genomes sequenced.

Conclusion.

Isolating tightly-bound peptides from the starch granule surface identified peptide sequences from six common field pathogens of wheat. Many other recovered peptides were unique in that they were the first occurrence of previous putative proteins indicated by genomic data. Recovering this novel community of both host and pathogen proteins clustered on the granule landscape permits a more dynamic tracking of the penetrative power of pathogens and their proteins.

These results demonstrate that the methods described herein are effective for characterizing host-pathogen interactions at the surface of starch granules isolated from plants, and that the methods are useful for detecting the exposure of plants to plant pathogens.

### Example 2

### Separation of Peptides Isolated from Fusarium-Infected Corn

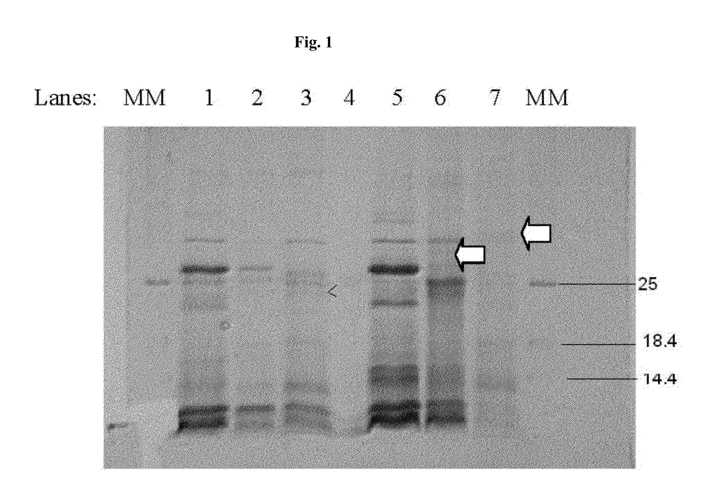

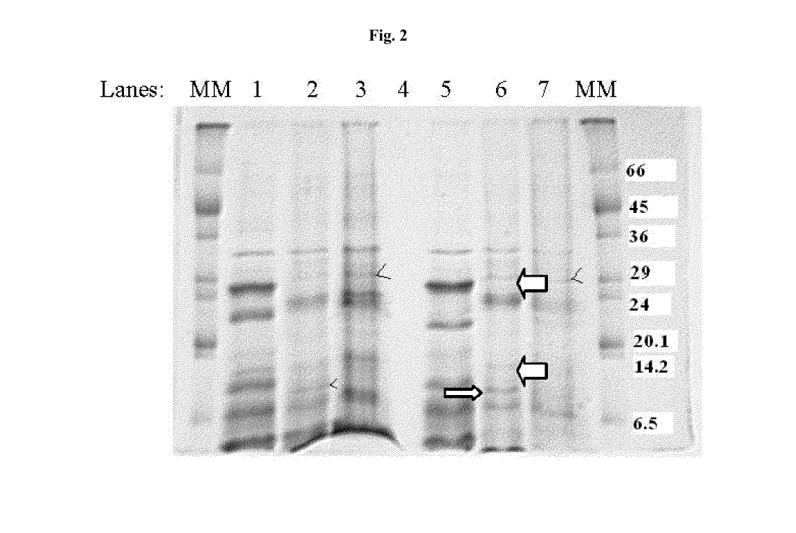

Maize kernels were collected from four agricultural testing sites and were prepared and analyzed as described above. Samples were collected from Sites 431, 359, 388, and C130 at the Central Experimental Farm, Agriculture Canada, Ottawa and infected with either F. verticillium or F. graminearum. Negative controls were infected with sterile water. Samples were prepared as described above and separated by SDS-PAGE and Coomassie stained. Extracts were adjusted to 1.0 mg/ml protein. For loading, 15 μl of each sample was mixed with 15 μl of loading buffer; 30 μl was loaded on the gel. Results are shown in FIG. 1 (Sites 431 and 388) and FIG. 2 (Sites 359 and C130). Arrows (<) indicate the position of proteins of interest present in the F. graminearum or F. verticillium kernel extracts and absent in the control samples. The arrows (<) indicate fungal protein bands present in the F. verticillium and F. graminearum kernel extracts but not in the control sample.

These results demonstrate that the exposure of corn to plant pathogens alters the profile of peptides present on the surface of starch granules, and that such alterations may be used as an indicator of corn exposure to plant pathogens.

### Example 3

### Isolation and Identification of Peptides from Starch Granules of Non-Grains

Starch Granule Extraction.

Non-grain plant samples are sterilized in 0.25% sodium hypochlorite (4% domestic bleach solution) for 10 min, rinsed with distilled water 20-30 times, and dried overnight in a laminar flow hood. Samples are ground into a meal or homogenate using a separate, clean mortar and pestle for each. The following procedure is performed in triplicate for each sample: an aliquot of the homogenized plant material (2 g) is mixed with distilled water to form a dough ball. Distilled water is passed through the dough ball, and through gentle squeezing of the dough ball, a starch granule-rich flow-through is obtained. To collect the granules, this flow-through is centrifuged at 1600×g for 2 minutes; the supernatant is discarded and the starch pellet is resuspended in distilled water. The resuspension and centrifugation wash step is repeated twenty times for each sample.

Sampling the Starch Granule Surface Proteome.

Extraction of peptides from the surface of starch granules is carried out in two phases. First, starch granules are treated with an aqueous solvent (“tryptic shaving”) comprising trypsin in order to remove peptides present on the outer-most surface layer (“distal peptides”). Second, starch granules are contacted with an organic solvent in order to remove more tightly bound, inner layer peptides (“proximal peptides”).

For tryptic shaving, aliquots of water-washed starch granules (approximately 150 mg) are subjected to enzymatic shaving with trypsin (Promega, Madison, Wis.; USA; 5 μg trypsin in 50 mM sodium bicarbonate, 37° C. overnight). Trypsin-treated granules are centrifuged (18000×g, 1 minute) and the aqueous supernatant transferred to a fresh tube. The insoluble starch pellet is washed three times in distilled water. Peptides remaining on the insoluble granules are extracted by suspending the granules in 12 μl 50% isopropanol (v/v), 50 mM NaCl per milligram wet weight. The granule suspensions are mixed at room temperature for 45 minutes and centrifuged at 2500×g for 5 minutes to obtain the isopropanol supernatant. Insoluble pellets are discarded. The aqueous and isopropanol supernatants are dried under vacuum at 30° C. for 4 h. Peptide pellets are resuspended in 40 μl of distilled water, purified using ZipTips® (Millipore, Bedford, Mass., USA) to remove salt and residual starch, and dried under vacuum at 30° C. for 1 hour. Peptides are resuspended in 40 μl of 0.1% formic acid (v/v), separated by high performance liquid chromatography (HPLC) and analyzed by electrospray ionization mass spectrometry (ESI-MS), as detailed below.

Chromatography and Mass Spectrometry.

Peptides are injected onto a 7 cm×200 μm inner diameter trap column, fitted, and packed in-house with 5 cm of 5 μm YMC ODS A reversed phase packing material (Waters, Milford, Mass.) using an Agilent 1100 HPLC (Agilent, Santa Clara, Calif., USA). The peptides are washed for 4 minutes at 20 μl/min with an aqueous solution containing 5% acetonitrile (v/v) and 0.1% formic acid (v/v). The trap column is connected in series to a 6 cm×75 μm Picofrit analytical column with a tip opening of 10 μm (New Objective, Woburn, Mass.), packed with 5 cm of YMC ODS A, for mass spectrometric analysis as well as a diverter valve to split the solvent flow prior to the columns. Peptides are eluted from the trap and analytical columns at a flow rate of ˜250 nl/min, ionized by nanoelectrospray ionization (ESI) and analyzed using a QSTAR® Pulsar QqTOF mass spectrometer (Applied Biosystems, Foster City, Calif., USA) operating in information-dependent acquisition mode. Mass analysis includes a 1 second survey scan followed by four 3 second tandem mass spectrometric scans on the most intense peaks in the spectrum; masses may be sequenced twice before being added to an exclusion list for 90 seconds. The HPLC pumps 0.1% formic acid (v/v) in water with the following percentage gradient of acetonitrile: 0 min: 5%, 3 min: 15%, 50 min: 40%, 55 min: 50% and 60 min: 80%. MS/MS data are searched against a custom database consisting of all plant proteins from the comprehensive, non-redundant protein database, NCBInr, using Mascot (Matrixscience Ltd, Boston, Mass., USA). Mass tolerances are set to ±100 ppm and ±0.2 Da for the peptide and fragment ion spectra, respectively; up to two missed cleavages are considered. Oxidation of methionine residues is selected as a variable modification; no fixed modifications are selected as the ‘tryptic shaving’ does not involve an alkylation step. Mass spectral significance is determined by searching the data against a “decoy” database containing a copy of the NCBInr database in reverse order and adjusting the ion score cutoff to ≧15 such that the false positive rate (proportional to the number of peptides matching to reversed sequences in the database) is equal to or less than 1%. Each match from the database is verified manually; data are deemed acceptable if at least three successive y- or b-ions are present (or y-++ or b-++ ions if the charge state of the peptide is greater than 2+).

Results.

It is expected that peptides identical to regions of proteins from common plant pathogens will be identified as having been present on the surfaces of starch granules. It is further expected that one or more of the peptides will be derived from one or more of the following species: F. graminearum, P. carotovora, X. oryzae, Agrobacterium tumefaciens, Magnaporthe grisea, and Xanthomonas axonopodis and Puccinia graminis f. and any of its subspecies such as Uganda strain 99 (Ug99). Examples of plant pathogen-derived peptides that may be identified by the present methods include but are not limited to the peptides given in Table 1. It is further expected that in addition to previously known proteins, one or more of the peptides identified by the present methods may derive from as yet unidentified plant pathogen proteins.

It is moreover expected that one or more of the peptides identified will be derived from the host plant. It is further expected that the one or more peptides may be involved in host defense against plant pathogens. Host-derived peptides that may be identified by the present methods include but are not limited to the peptides given in Tables 2-5. It is further expected that in addition to previously known proteins, one or more of the peptides identified by the present methods may derive from as yet unidentified plant proteins.

These results demonstrate that the methods described herein are effective for characterizing host-pathogen interactions at the surface of starch granules isolated from non-grains, and demonstrate that the methods are useful for detecting the exposure of non-grains to plant pathogens.

### Example 4

### Isolation and Identification of Peptides from Starch Granules of Fruit

This Example will demonstrate methods for the isolation and identification of peptides from starch granules of fruits by methods described herein.

Starch Granule Extraction.

Fruits are peeled and cut into thin slices. Representative samples of 500 g from each genetic variety are blended with an extractive solvent (0.05 N NaOH) at a ratio 500 grams per kilogram of solvent. The fruit and solvent mixture is blended in a Waring blender for 2 minutes and then stirred slowly for 2 hours. The resulting suspension is centrifuged at 1,000×g. The sediment is resuspended in two volumes of distilled water and sieved through 200 and 325 mesh screen or cloth, until the washing water is clear. The starch suspension is then neutralized to pH 6.2 (range 5.2-7.2) and the sediment starch is dried overnight in a laminar flow hood at ambient temperature or in a circulating air oven at up to 50° C. The dried granule preparation can be ground with a centrifugal mill (Retsch, ZM100) to pass through a 100 mesh sieve (Retsch, VE1000) and stored at room temperature in sealed plastic containers until analyzed by the methods described above.

Sampling the Starch Granule Surface Proteome.

Extraction of peptides from the surface of starch granules is carried out in two phases. First, starch granules are treated with an aqueous solvent (“tryptic shaving”) comprising trypsin in order to remove peptides present on the outer-most surface layer (“distal peptides”). Second, starch granules are contacted with an organic solvent in order to remove more tightly bound, inner layer peptides (“proximal peptides”).

For tryptic shaving, aliquots of water-washed starch granules (approximately 150 mg) are subjected to enzymatic shaving with trypsin (Promega, Madison, Wis.; USA; 5 μg trypsin in 50 mM sodium bicarbonate, 37° C. overnight). Trypsin-treated granules are centrifuged (18000×g, 1 minute) and the aqueous supernatant transferred to a fresh tube. The insoluble starch pellet is washed three times in distilled water. Peptides remaining on the insoluble granules are extracted by suspending the granules in 12 μl 50% isopropanol (v/v), 50 mM NaCl per milligram wet weight. The granule suspensions are mixed at room temperature for 45 minutes and centrifuged at 2500×g for 5 minutes to obtain the isopropanol supernatant. Insoluble pellets are discarded. The aqueous and isopropanol supernatants are dried under vacuum at 30° C. for 4 h. Peptide pellets are resuspended in 40 μl of distilled water, purified using ZipTips® (Millipore, Bedford, Mass., USA) to remove salt and residual starch, and dried under vacuum at 30° C. for 1 hour. Peptides are resuspended in 40 μl of 0.1% formic acid (v/v), separated by high performance liquid chromatography (HPLC) and analyzed by electrospray ionization mass spectrometry (ESI-MS), as detailed below.

Chromatography and Mass Spectrometry.

Peptides are injected onto a 7 cm×200 μm inner diameter trap column, fitted, and packed in-house with 5 cm of 5 μm YMC ODS A reversed phase packing material (Waters, Milford, Mass.) using an Agilent 1100 HPLC (Agilent, Santa Clara, Calif., USA). The peptides are washed for 4 minutes at 20 μl/min with an aqueous solution containing 5% acetonitrile (v/v) and 0.1% formic acid (v/v). The trap column is connected in series to a 6 cm×75 μm Picofrit analytical column with a tip opening of 10 μm (New Objective, Woburn, Mass.), packed with 5 cm of YMC ODS A, for mass spectrometric analysis as well as a diverter valve to split the solvent flow prior to the columns. Peptides are eluted from the trap and analytical columns at a flow rate of ˜250 nl/min, ionized by nanoelectrospray ionization (ESI) and analyzed using a QSTAR® Pulsar QqTOF mass spectrometer (Applied Biosystems, Foster City, Calif., USA) operating in information-dependent acquisition mode. Mass analysis includes a 1 second survey scan followed by four 3 second tandem mass spectrometric scans on the most intense peaks in the spectrum; masses may be sequenced twice before being added to an exclusion list for 90 seconds. The HPLC pumps 0.1% formic acid (v/v) in water with the following percentage gradient of acetonitrile: 0 min: 5%, 3 min: 15%, 50 min: 40%, 55 min: 50% and 60 min: 80%. MS/MS data are searched against a custom database consisting of all plant proteins from the comprehensive, non-redundant protein database, NCBInr, using Mascot (Matrixscience Ltd, Boston, Mass., USA). Mass tolerances are set to ±100 ppm and ±0.2 Da for the peptide and fragment ion spectra, respectively; up to two missed cleavages are considered. Oxidation of methionine residues is selected as a variable modification; no fixed modifications are selected as the ‘tryptic shaving’ does not involve an alkylation step. Mass spectral significance is determined by searching the data against a “decoy” database containing a copy of the NCBInr database in reverse order and adjusting the ion score cutoff to ≧15 such that the false positive rate (proportional to the number of peptides matching to reversed sequences in the database) is equal to or less than 1%. Each match from the database is verified manually; data are deemed acceptable if at least three successive y- or b-ions are present (or y-++ or b-++ ions if the charge state of the peptide is greater than 2+).

Results.

It is expected that peptides identical to regions of proteins from common plant pathogens will be identified as having been present on the surfaces of starch granules. It is further expected that one or more of the peptides will be derived from one or more of the following species: F. graminearum, P. carotovora, X. oryzae, Agrobacterium tumefaciens, Magnaporthe grisea, and Xanthomonas axonopodis and Puccinia graminis f. and any of its subspecies such as Uganda strain 99 (Ug99). Examples of plant pathogen-derived peptides that may be identified by the present methods include but are not limited to the peptides given in Table 1. It is further expected that in addition to previously known proteins, one or more of the peptides identified by the present methods may derive from as yet unidentified plant pathogen proteins.

It is moreover expected that one or more of the peptides identified will be derived from the host plant. It is further expected that the one or more peptides may be involved in host defense against plant pathogens. Host-derived peptides that may be identified by the present methods include but are not limited to the peptides given in Tables 2-5. It is further expected that in addition to previously known proteins, one or more of the peptides identified by the present methods may derive from as yet unidentified plant proteins.

These results demonstrate that the methods described herein are effective for characterizing host-pathogen interactions at the surface of starch granules isolated from fruits, and demonstrate that the methods are useful for detecting the exposure of fruits to plant pathogens.

### EQUIVALENTS

While certain embodiments have been illustrated and described, it should be understood that changes and modifications can be made therein in accordance with ordinary skill in the art without departing from the technology in its broader aspects as defined in the following claims.

The embodiments, illustratively described herein, may suitably be practiced in the absence of any element or elements, limitation or limitations, not specifically disclosed herein. Thus, for example, the terms “comprising,” “including,” “containing,” etc., shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detecting the exposure of a non-grain plant or plant part to at least one plant pathogen, the method comprising:
(a) isolating at least one starch granule from the non-grain plant or plant part;
(b) contacting the starch granule with at least one solvent; wherein the contacting releases at least one peptide from the surface of the starch granule; and
(c) determining the amino acid sequence of the peptide;
wherein the presence of at least one peptide from a plant pathogen indicates that the non-grain has been exposed to at least one plant pathogen.

2. The method of claim 1, wherein the non-grain comprises fruits, nuts, vegetables, legumes, tubers, leaves, trees, flowers, herbs, bushes, grasses, vines, ferns, mosses, fungi, petals, flour, anthers, pollen, pollen grains, glumes, sugarcane stems, awns, straw, grain dust, palea, pedicels, rachilla, lodicules, stolons, rhizomes, sheaths, collars, blades, auricles, ligules, culms, or green algae.

3. The method of claim 1, wherein the solvent comprises at least one protease.

4. The method of claim 3, wherein the protease comprises trypsin or chymotrypsin.

5. The method of claim 1, wherein the at least one plant pathogen comprises a viral pathogen, a bacterial pathogen, or a fungal pathogen.

6. The method of claim 1, wherein the at least one plant pathogen comprises Agrobacterium, Pectobacterium, Fusarium, Magnaporthe, or Xanthomonas.

7. The method of claim 1, wherein the at least one plant pathogen comprises Agrobacterium tumefaciens, Pectobacterium carotovorum, Fusarium graminearum, Magnaporthe grisea, Xanthomonas axonopodis, or Xanthomonas oryzae.

8. The method of claim 1, wherein the determining step comprises chromatography, mass spectrometry, or both.

9. The method of claim 1, wherein the amino acid sequences of the peptide comprise at least one amino acid sequence selected from the group consisting of SEQ ID NOs: 1-108.

10. A method for detecting a response to at least one plant pathogen in a non-grain plant or plant part, the method comprising:
(a) isolating at least one starch granule from the non-grain plant or plant part;
(b) contacting the starch granule with at least one solvent; wherein the contacting releases at least one peptide from the surface of the starch granule; and
(c) determining the amino acid sequence of the peptide;
wherein the presence of at least one peptide from non-grain anti-microbial proteins indicates a non-grain response to at least one plant pathogen.

11. The method of claim 10, wherein the non-grain comprises fruits, nuts, vegetables, legumes, tubers, leaves, trees, flowers, herbs, bushes, grasses, vines, ferns, mosses, fungi, or green algae.

12. The method of claim 10, wherein the solvent comprises at least one protease.

13. The method of claim 12, wherein the protease comprises trypsin or chymotrypsin.

14. The method of claim 10, wherein the at least one plant pathogen comprises a viral pathogen, a bacterial pathogen, or a fungal pathogen.

15. The method of claim 10, wherein the at least one plant pathogen comprises Agrobacterium, Pectobacterium, Fusarium, Magnaporthe, or Xanthomonas.

16. The method of claim 10, wherein the at least one plant pathogen comprises Agrobacterium tumefaciens, Pectobacterium carotovorum, Fusarium graminearum, Magnaporthe grisea, Xanthomonas axonopodis, or Xanthomonas oryzae.

17. The method of claim 10, wherein the determining step comprises chromatography, mass spectrometry, or both.

18. The method of claim 10, wherein the amino acid sequences of the peptide comprise at least one amino acid sequence selected from the group consisting of SEQ ID NOs: 1-108.

19. A kit for detecting the exposure of a non-grain plant or plant part to at least one plant pathogen, the kit comprising:
(a) at least one solvent, wherein the at least one solvent extracts peptides present on the surface of at least one starch granule isolated from the non-grain;
(b) at least one negative control sample, comprising at least one starch granule from a pest-free, pathogen-free non-grain; and
(c) at least one positive control, comprising at least one starch granule isolated from a plant exposed to a plant pathogen.

20. The kit of claim 19, wherein the negative control sample comprises starch from a plant resistant to Fusarium.

21. The kit of claim 19, wherein the positive control sample comprises starch from a plant contacted with Fusarium.

22. The kit of claim 19, wherein the positive control sample comprises at least one peptide having an amino acid sequence selected from the group consisting of SEQ ID NOs: 1-108.

23. The kit of claim 19, wherein the solvent comprises trypsin or chymotrypsin.

24-27. (canceled)


SEQUENCE LISTING (108 sequences)

SEQ ID NO: 1 - protein, 14 aa, Unknown
  Description of Unknown Hard wheat peptide sequence
KLDDPAGYGRIVRE

SEQ ID NO: 2 - protein, 14 aa, Unknown
  Description of Unknown Hard wheat peptide sequence
RDRVADAWPVFCKY

SEQ ID NO: 3 - protein, 11 aa, Unknown
  Description of Unknown Hard wheat peptide sequence
RALGKSKNART

SEQ ID NO: 4 - protein, 10 aa, Unknown
  Description of Unknown Hard wheat peptide sequence
RATLANRKRV

SEQ ID NO: 5 - protein, 14 aa, Unknown
  Description of Unknown Hard wheat peptide sequence
RVRAEMVAKMDEKG

SEQ ID NO: 6 - protein, 11 aa, Unknown
  Description of Unknown Hard wheat peptide sequence
KVVDTAAKKRG

SEQ ID NO: 7 - protein, 14 aa, Unknown
  Description of Unknown Hard wheat peptide sequence
KASRVRDSQRASRA

SEQ ID NO: 8 - protein, 10 aa, Unknown
  Description of Unknown Hard wheat peptide sequence
KIAMSELARR

SEQ ID NO: 9 - protein, 16 aa, Unknown
  Description of Unknown Hard wheat peptide sequence
KACPTAEMRGVALGRF

SEQ ID NO: 10 - protein, 13 aa, Unknown
  Description of Unknown Hard wheat peptide sequence
KKPTAQEIAITRG

SEQ ID NO: 11 - protein, 10 aa, Unknown
  Description of Unknown Hard wheat peptide sequence
RVVVMQDGRI

SEQ ID NO: 12 - protein, 19 aa, Unknown
  Description of Unknown Hard wheat peptide sequence
RLSITPAAVSRNVAMLERN

SEQ ID NO: 13 - protein, 24 aa, Unknown
  Description of Unknown Hard wheat peptide sequence
RAREVVIATGPWSPALAAQLGLRL

SEQ ID NO: 14 - protein, 10 aa, Unknown
  Description of Unknown Soft wheat peptide sequence
RMRKTLLARH

SEQ ID NO: 15 - protein, 10 aa, Unknown
  Description of Unknown Soft wheat peptide sequence
MNIPVPVVRL

SEQ ID NO: 16 - protein, 10 aa, Unknown
  Description of Unknown Hard wheat peptide sequence
KLTNVALGRQ

SEQ ID NO: 17 - protein, 12 aa, Unknown
  Description of Unknown Hard wheat peptide sequence
RADNLYWEGFKS

SEQ ID NO: 18 - protein, 15 aa, Unknown
  Description of Unknown Hard wheat peptide sequence
KTSPASTGLTLATRA

SEQ ID NO: 19 - protein, 17 aa, Unknown
  Description of Unknown Hard wheat peptide sequence
RFQTVSGFVAGVLEPKE

SEQ ID NO: 20 - protein, 17 aa, Unknown
  Description of Unknown Hard wheat peptide sequence
KAQVNGWQDLSEALLKT

SEQ ID NO: 21 - protein, 17 aa, Unknown
  Description of Unknown Hard wheat peptide sequence
REAVTTLLLMVHEATRF

SEQ ID NO: 22 - protein, 18 aa, Unknown
  Description of Unknown Hard wheat peptide sequence
RDLLGDTDKLTNVALGRQ

SEQ ID NO: 23 - protein, 29 aa, Unknown
  Description of Unknown Hard wheat peptide sequence
KSSDGTWWELTPGLIPGATHVGFGGTYRD

SEQ ID NO: 24 - protein, 13 aa, Unknown
  Description of Unknown Hard wheat peptide sequence
KVLSDASAFIWKT

SEQ ID NO: 25 - protein, 16 aa, Unknown
  Description of Unknown Hard wheat peptide sequence
KSGYTDDFFAQILGKN

SEQ ID NO: 26 - protein, 24 aa, Unknown
  Description of Unknown Hard wheat peptide sequence
RAIGVDLLSNPDLVATDPTVSFKT

SEQ ID NO: 27 - protein, 12 aa, Unknown
  Description of Unknown Hard wheat peptide sequence
KDIGLAAGLLRL

SEQ ID NO: 28 - protein, 14 aa, Unknown
  Description of Unknown Hard wheat peptide sequence
RDSVVVSGGPDYRV

SEQ ID NO: 29 - protein, 24 aa, Unknown
  Description of Unknown Hard wheat peptide sequence
REGLFVSDQDLFTNDITRPIVERF

SEQ ID NO: 30 - protein, 17 aa, Unknown
  Description of Unknown Hard wheat peptide sequence
RFQTVSGFVAGLLHPKA

SEQ ID NO: 31 - protein, 10 aa, Unknown
  Description of Unknown Hard wheat peptide sequence
LPVLAPVTKD

SEQ ID NO: 32 - protein, 18 aa, Unknown
  Description of Unknown Soft wheat peptide sequence
RDLLGDTDKLTNVALGRQ

SEQ ID NO: 33 - protein, 16 aa, Unknown
  Description of Unknown Soft wheat peptide sequence
KSGYTDDFFAQILGKN

SEQ ID NO: 34 - protein, 24 aa, Unknown
  Description of Unknown Soft wheat peptide sequence
RAIGVDLLSNPDLVATDPTVSFKT

SEQ ID NO: 35 - protein, 12 aa, Unknown
  Description of Unknown Soft wheat peptide sequence
KDIGLAAGLLRL

SEQ ID NO: 36 - protein, 24 aa, Unknown
  Description of Unknown Soft wheat peptide sequence
REGLFVSDQDLFTNDITRPIVERF

SEQ ID NO: 37 - protein, 11 aa, Unknown
  Description of Unknown Soft wheat peptide sequence
RALATGIFERA

SEQ ID NO: 38 - protein, 9 aa, Unknown
  Description of Unknown Soft wheat peptide sequence
KSAQKTERF

SEQ ID NO: 39 - protein, 12 aa, Unknown
  Description of Unknown Soft wheat peptide sequence
KVPIPNPSGDRA

SEQ ID NO: 40 - protein, 11 aa, Unknown
  Description of Unknown Soft wheat peptide sequence
KDFPVTWPTKW

SEQ ID NO: 41 - protein, 15 aa, Unknown
  Description of Unknown Hard wheat peptide sequence
RQQMADAVTALYGRT

SEQ ID NO: 42 - protein, 17 aa, Unknown
  Description of Unknown Hard wheat peptide sequence
RFQTVSGFVAGVLHPKE

SEQ ID NO: 43 - protein, 34 aa, Unknown
  Description of Unknown Hard wheat peptide sequence
RTAEQAAATLGILLFVQVPGGMTVAQALELFHKS

SEQ ID NO: 44 - protein, 15 aa, Unknown
  Description of Unknown Soft wheat peptide sequence
KTSPASTGLTLATRA

SEQ ID NO: 45 - protein, 15 aa, Unknown
  Description of Unknown Soft wheat peptide sequence
RQQMADAVTALYGRT

SEQ ID NO: 46 - protein, 17 aa, Unknown
  Description of Unknown Soft wheat peptide sequence
KAQVNGWQDLSEALLKT

SEQ ID NO: 47 - protein, 18 aa, Unknown
  Description of Unknown Soft wheat peptide sequence
RGPIQLSHNYNYGPAGRA

SEQ ID NO: 48 - protein, 16 aa, Unknown
  Description of Unknown Hard wheat peptide sequence
RANGGLIEGIADYVRL

SEQ ID NO: 49 - protein, 16 aa, Unknown
  Description of Unknown Hard wheat peptide sequence
RVPTVDVSVVDLTVRI

SEQ ID NO: 50 - protein, 12 aa, Unknown
  Description of Unknown Hard wheat peptide sequence
RVLIVVNTEERK

SEQ ID NO: 51 - protein, 14 aa, Unknown
  Description of Unknown Hard wheat peptide sequence
MLAFPLLELGQMKE

SEQ ID NO: 52 - protein, 16 aa, Unknown
  Description of Unknown Hard wheat peptide sequence
RNTAGEEKQQTLALRV

SEQ ID NO: 53 - protein, 10 aa, Unknown
  Description of Unknown Hard wheat peptide sequence
KSCLMPLRKS

SEQ ID NO: 54 - protein, 14 aa, Unknown
  Description of Unknown Hard wheat peptide sequence
KFDRVESEVALLRE

SEQ ID NO: 55 - protein, 9 aa, Unknown
  Description of Unknown Hard wheat peptide sequence
RSAKPQHRI

SEQ ID NO: 56 - protein, 13 aa, Unknown
  Description of Unknown Hard wheat peptide sequence
MVEMMRSENHLRQ

SEQ ID NO: 57 - protein, 15 aa, Unknown
  Description of Unknown Hard wheat peptide sequence
KCDNMASQYFVCCRW

SEQ ID NO: 58 - protein, 17 aa, Unknown
  Description of Unknown Hard wheat peptide sequence
KTPPPPPPPPPPLPSRS

SEQ ID NO: 59 - protein, 13 aa, Unknown
  Description of Unknown Hard wheat peptide sequence
RIVVADPPYLSKE

SEQ ID NO: 60 - protein, 17 aa, Unknown
  Description of Unknown Hard wheat peptide sequence
KQVEEMVLVKWVEWKKE

SEQ ID NO: 61 - protein, 9 aa, Unknown
  Description of Unknown Hard wheat peptide sequence
RIPRWRRRL

SEQ ID NO: 62 - protein, 12 aa, Unknown
  Description of Unknown Hard wheat peptide sequence
KCSIAKDKPKRN

SEQ ID NO: 63 - protein, 16 aa, Unknown
  Description of Unknown Soft wheat peptide sequence
RANGGLIEGIADYVRL

SEQ ID NO: 64 - protein, 10 aa, Unknown
  Description of Unknown Soft wheat peptide sequence
RAIGVDLLRN

SEQ ID NO: 65 - protein, 16 aa, Unknown
  Description of Unknown Soft wheat peptide sequence
RNPDLVATDPTVSFKT

SEQ ID NO: 66 - protein, 15 aa, Unknown
  Description of Unknown Soft wheat peptide sequence
RSQAKAVAPTSRARS

SEQ ID NO: 67 - protein, 12 aa, Unknown
  Description of Unknown Soft wheat peptide sequence
REAASLAEAAKD

SEQ ID NO: 68 - protein, 10 aa, Unknown
  Description of Unknown Soft wheat peptide sequence
RVVESMRSKS

SEQ ID NO: 69 - protein, 12 aa, Unknown
  Description of Unknown Soft wheat peptide sequence
RQLVQIPEQARC

SEQ ID NO: 70 - protein, 9 aa, Unknown
  Description of Unknown Soft wheat peptide sequence
RCSAKYLKS

SEQ ID NO: 71 - protein, 10 aa, Unknown
  Description of Unknown Soft wheat peptide sequence
KVVTVSARPK

SEQ ID NO: 72 - protein, 10 aa, Unknown
  Description of Unknown Soft wheat peptide sequence
RASKSGLHRV

SEQ ID NO: 73 - protein, 13 aa, Unknown
  Description of Unknown Soft wheat peptide sequence
RVQKASISLVMKL

SEQ ID NO: 74 - protein, 13 aa, Unknown
  Description of Unknown Soft wheat peptide sequence
KNPLAIVNVKGRA

SEQ ID NO: 75 - protein, 14 aa, Unknown
  Description of Unknown Hard wheat peptide sequence
KDAWDTSVVSEIKV

SEQ ID NO: 76 - protein, 15 aa, Unknown
  Description of Unknown Hard wheat peptide sequence
KEALQAEVGLPVDRK

SEQ ID NO: 77 - protein, 17 aa, Unknown
  Description of Unknown Hard wheat peptide sequence
KFLAANYDVTTALEGKA

SEQ ID NO: 78 - protein, 18 aa, Unknown
  Description of Unknown Hard wheat peptide sequence
KSSFDFIDGYDKPVEGRK

SEQ ID NO: 79 - protein, 20 aa, Unknown
  Description of Unknown Hard wheat peptide sequence
KVLTVSPYYAEELISGEARG

SEQ ID NO: 80 - protein, 13 aa, Unknown
  Description of Unknown Hard wheat peptide sequence
RYDPTAYNTILRN

SEQ ID NO: 81 - protein, 16 aa, Unknown
  Description of Unknown Hard wheat peptide sequence
RFFVDNGTYLTEQGRF

SEQ ID NO: 82 - protein, 17 aa, Unknown
  Description of Unknown Hard wheat peptide sequence
KAAAAMVGHPEWEFPRD

SEQ ID NO: 83 - protein, 18 aa, Unknown
  Description of Unknown Hard wheat peptide sequence
KSAPEELVQQVLSAGWRE

SEQ ID NO: 84 - protein, 20 aa, Unknown
  Description of Unknown Hard wheat peptide sequence
RNIEYLTLGVDDQPLFHGRT

SEQ ID NO: 85 - protein, 11 aa, Unknown
  Description of Unknown Hard wheat peptide sequence
RVAIMEVNPRA

SEQ ID NO: 86 - protein, 11 aa, Unknown
  Description of Unknown Hard wheat peptide sequence
RVAIMEVNPRA

SEQ ID NO: 87 - protein, 13 aa, Unknown
  Description of Unknown Hard wheat peptide sequence
RDTFNLLEQRPKI

SEQ ID NO: 88 - protein, 12 aa, Unknown
  Description of Unknown Hard wheat peptide sequence
KEGEGVIVLLRG

SEQ ID NO: 89 - protein, 17 aa, Unknown
  Description of Unknown Hard wheat peptide sequence
RTTTRVPFGVGTGVGGY

SEQ ID NO: 90 - protein, 13 aa, Unknown
  Description of Unknown Hard wheat peptide sequence
KIVAILGSMRVPN

SEQ ID NO: 91 - protein, 14 aa, Unknown
  Description of Unknown Hard wheat peptide sequence
KAPFASIVADIGGQ

SEQ ID NO: 92 - protein, 16 aa, Unknown
  Description of Unknown Hard wheat peptide sequence
KAIWTSIQGDLSGFKG

SEQ ID NO: 93 - protein, 10 aa, Unknown
  Description of Unknown Hard wheat peptide sequence
KVLRSRSRRL

SEQ ID NO: 94 - protein, 11 aa, Unknown
  Description of Unknown Hard wheat peptide sequence
RSKVTSFIHRM

SEQ ID NO: 95 - protein, 12 aa, Unknown
  Description of Unknown Hard wheat peptide sequence
RQLPQIPEQSRY

SEQ ID NO: 96 - protein, 15 aa, Unknown
  Description of Unknown Hard wheat peptide sequence
KTLWSRAILSKAVKL

SEQ ID NO: 97 - protein, 10 aa, Unknown
  Description of Unknown Hard wheat peptide sequence
RIYVVTSPRD

SEQ ID NO: 98 - protein, 25 aa, Unknown
  Description of Unknown Hard wheat peptide sequence
RLLAGVTIAHGGVIPNINSVLLPKK

SEQ ID NO: 99 - protein, 14 aa, Unknown
  Description of Unknown Hard wheat peptide sequence
KISFGIEASDLLKC

SEQ ID NO: 100 - protein, 12 aa, Unknown
  Description of Unknown Soft wheat peptide sequence
KEGEGVIVLLRG

SEQ ID NO: 101 - protein, 10 aa, Unknown
  Description of Unknown Soft wheat peptide sequence
KVFSDEVIRF

SEQ ID NO: 102 - protein, 13 aa, Unknown
  Description of Unknown Soft wheat peptide sequence
KKIWSKDIPPSKS

SEQ ID NO: 103 - protein, 13 aa, Unknown
  Description of Unknown Soft wheat peptide sequence
RRELESGKEEGRR

SEQ ID NO: 104 - protein, 11 aa, Unknown
  Description of Unknown Soft wheat peptide sequence
RGMPWWVPFRS

SEQ ID NO: 105 - protein, 11 aa, Unknown
  Description of Unknown Soft wheat peptide sequence
RGMPWWVPFRS

SEQ ID NO: 106 - protein, 13 aa, Unknown
  Description of Unknown Soft wheat peptide sequence
KDPVPVNTRRSKF

SEQ ID NO: 107 - protein, 14 aa, Unknown
  Description of Unknown Soft wheat peptide sequence
KKVPLVLIQTSHRW

SEQ ID NO: 108 - protein, 11 aa, Unknown
  Description of Unknown Soft wheat peptide sequence
RNFKLTLIARR